(19) United States
(12) Patent Application Publication
(10) Pub. No.: US 2015/0119352 A1
(43) Pub. Date: Apr. 30, 2015

(54) DINUCELOSIDE POLYPHOSPHATES FOR THE TREATMENT OF PAIN

(71) Applicant: GlobalAcorn Ltd., London (GB)

(72) Inventors: Andrew David Miller, Chiswick (GB); Natalya Lozovaya, Marseille (FR); Nail Burnashev, Marseille (FR); Rashid Giniatullin, Kuopio (FI)

(73) Assignee: GlobalAcornLtd., London (GB)

(21) Appl. No.: 14/403,560

(22) PCT Filed: May 24, 2013
(86) PCT No.: PCT/GB2013/051377

§ 371 Date: Nov. 24, 2014

(30) Foreign Application Priority Data
May 25, 2012 (GB) 1209244.1

(51) Int. Cl. C07H 19/20; A61K 31/675; C07F 9/6561; A61K 31/7084

(52) U.S. Cl. CPC C07H 19/20; A61K 31/7084; A61K 31/675; C07F 9/65616
USPC 514/47; 536/26.22; 514/81; 544/244

(57) ABSTRACT

The invention provides a dinucleoside polyphosphate analogue, or a pharmaceutically acceptable salt thereof, for use in the inhibition (or down-regulation) of a pain, via a transducing ATP-gated P2X3 receptor, often by means of high-affinity desensitisation (HAD) mechanism.

### FIELD OF THE INVENTION

The present invention relates to the use of (analogues) of dinucleoside polyphosphates and other compounds as potent and selective inhibitors or down-regulators of P2X3 receptors, in particular to their use for the treatment (or prevention or reduction) of (acute to chronic) nociceptive pain, such as back pain.

### BACKGROUND TO THE INVENTION

More than 270 million people worldwide suffer from chronic pain, which is still treated predominantly by opioids and non-steroidal anti-inflammatory drugs (NSAIDs). While there have been small improvements in both these areas, they still suffer from significant adverse side effects and dependency issues.

It is suggested that P2X3 receptors are involved in various states of chronic pain, including inflammatory and cancer-associated pain. Previous studies have shown that P2X3 antagonists or genetic deletion can have analgesic effects on inflammatory and neuropathic pain models. The activities of P2X3 receptors may be inhibited by several non-nucleotide antagonists. AF-353, a bacterial DHFR inhibitor, is a potent and selective non competitive antagonist of P2X3 (Gever et al, 2010). It has been shown to allosterically modulate the interaction of nucleic acids with P2X3 without being a competitive antagonist of α,β-meATP. A-317491 is a competitive antagonist of P2X3 and P2×23, and binds to P2X3 receptors within a micromolar range of concentration (Jarvis et al, 2002). Systemic administration of A-317491 effectively reduced nociception in inflammatory and neuropathic pain models (Jarvis et al., 2002; McGaraughty et al., 2003). A-317491 also effectively blocked persistent pain in the formalin and acetic acid-induced abdominal constriction tests but was generally inactive in models of acute noxious stimulation. A-317491 is more efficient when injected intrathecally than in peripheral nervous system (Jarvis et al, 2002), indicating action within the central nervous system. RO-3, a non-competitive antagonist of P2X3 receptors, has been found to induce anti-nociception in animal models (Gever et al., 2006). Purotoxin-1, a spider venom peptidic toxin, binds to P2X3 and exerts a selective inhibitory action on P2X3 receptors (Grishin et al., 2010), its binding mechanism is not well known.

However research into potent P2X3-selective ligands with reasonable bioavailability is still lacking. To date, no selective P2X3 receptor antagonists have been evaluated successfully in clinic for the relief of chronic nociceptive or neuropathic pain.

### SUMMARY OF THE INVENTION

The present invention represents a useful and potent alternative to existing P2X3 ligands and can alleviate (some of) the problems of the prior art.

In one aspect, the present invention provides a dinucleoside polyphosphate (analogue), or a pharmaceutically acceptable salt thereof, for use in the (e.g. selective) inhibition (or down-regulation) of a pain transducing ATP-gated P2X3 receptor. In particular, the dinucleoside polyphosphate analogue may be for use in the treatment of pain, especially moderate to chronic pain, or back pain. Thus, the present invention also provides a dinucleoside polyphosphate (analogue), or a pharmaceutically acceptable salt thereof, for use in the treatment of (moderate to chronic) pain.

In another aspect, the present invention provides a method of inhibition (or down-regulation) of a pain transducing ATP-gated P2X3 receptor comprising administering an effective amount of a dinucleoside polyphosphate (analogue) or a pharmaceutically acceptable salt thereof.

The invention further provides the use of a dinucleoside polyphosphate analogue or a pharmaceutically acceptable salt thereof in the manufacture of a medicament for inhibition (or down-regulation) of a pain transducing ATP-gated P2X3 receptor.

The invention also relates to compounds, such as dinucleoside phosphates, that can act on selected receptor(s) and/or through novel mechanisms (such as HAD).

The dinucleoside polyphosphate analogues as used in the present invention are particularly potent, and are effective in the inhibition of pain (especially moderate to chronic pain) when administered at low concentrations.

### BRIEF DESCRIPTION OF THE DRAWINGS

FIG. 1. Partial agonist activity of AppNHppA at rat homomeric P2X3 receptors. A. Example of responses induced by different concentrations of AppNHppA (1 μM-100 μM) applied for 2 s to HEK cells expressing rat P2X3 receptors. B. Comparison of currents induced by the full agonist of P2X3 receptors α,β-meATP (10 μM, left) and saturating (1 mM, right) concentration of AppNHppA in the same cell. Note the significantly smaller amplitude of the latter. C. Bar graphs showing mean±SEM amplitude of currents induced by α,β-meATP at 10 μM and AppNHppA at different concentrations (10 μM 1 mM), n=7.

Here and below *p<0.05, **p<0.001, ***p<0.0001.

FIG. 2. Activation and inhibition of rat P2X3 receptors by AppNHppA. Top: Experimental protocol. Test pulses of 10 μM of α,β-meATP were delivered every 2 min After two control pulses, AppNHppA at different concentrations was applied for 6 minutes. A, B. Example traces of P2X3 mediated currents in control and after 2 min application of 10 nM (A) and 3 μM (B) of AppNHppA (corresponding to the part of the experimental protocol outlined by a dotted box). C. The time course of the current inhibition by AppNHppA at different (1 nM-3 μM) concentrations (n=4-6). Current amplitudes are normalized to control. D. Dose-response curves for activation and inhibition of P2X3 mediated currents by AppNHppA. Note that inhibitory curve for AppNHppA (IC₅₀=0.2±0.04 μM, n=3-6) does not overlap with the dose-response curve for activation (EC₅₀=41.6±1.3 μM; n_{H}=1.58±0.05; n=8).

FIG. 3. The inhibitory and agonist effect of AppCH₂ppA on homomeric P2X3 receptors. A. Example traces of P2X3 mediated currents in control and after application of 1 μM AppCH₂ppA. The same experimental protocol as shown in FIG. 2. Test 10 μM α,β-meATP pulses were applied every 2 mM (low rate). B. The same as in A, but test α,β-meATP pulses were applied every 30 s (high rate). C. Bar graphs showing inhibitory action of AppCH₂ppA at low and high rate of activation. D. Dose-response curves for activation and inhibition of P2X3 mediated currents by AppCH₂ppA. IC₅₀=0.55±0.2 μM, (n=4-7), EC₅₀=9.30±1.7 μM; n_{H}=2.27±0.56; (n=5).

FIG. 4. The non-inhibitory effect of AppNHppA on homomeric P2X2, P2X4 and P2X7 receptors. A. Example traces of responses induced by 10 μM ATP applied for 2 s every 2 min to HEK cells expressing rat homomeric P2X2 receptors in the presence and absence of 1 μM AppNHppA. B. Comparison of currents induced by the full agonist of P2X2 receptors, ATP, in the presence of 1 μM AppNHppA (right) and absence (left). C. The time course of the current in the presence and absence of 1 μM AppNHppA. D. Comparison of currents induced by the full agonist of rat homomeric P2X4 and P2X7 receptors, ATP, in the presence of 1 μM AppNHppA (right) and absence (left). Experiments were performed as in FIG. 2.

FIG. 5. Selective inhibition of P2X3 subunit containing receptors by AppNHppA in rat sensory neurons. A. The relative proportion of cells with fast, mixed and slow current types elicited by α,β-meATP in TG, DRG or NG neurons. Typical examples of fast, mixed and slow currents are shown in panels B-D respectively. B-D. Examples showing inhibitory action of AppNHppA (1 μM, 6 min application) on responses evoked by 10 μM α,β-meATP in trigeminal (TG), dorsal root (DRG) and nodose (NG) ganglia neurons, respectively. Note preferential inhibition of fast currents. E-G. Bar graphs showing the inhibitory action of AppNHppA on fast (peak) and slow (residual, at the end of α,β-meATP application) currents in different ganglia in TG (E), DRG (F) or NG (G) neurons.

FIG. 6. Effects of subcutaneous Ap₄A analogues on pain responses induced by intraplantar (right hindpaw) formalin injection. Rats were subcutaneously injected with formalin solution alone (0.5%, 50 μL, control) or co-injected with the formalin solution and solutions with AppCH₂ppA (1 μM-100 μM, 100 μL) or AppNHppA (0.1 μM-100 μM, 100 μL). Age-matched control animals were injected with saline. A. Time-course of the effects of subcutaneous AppCH₂ppA (a) and AppNHppA (b) on the number of the spontaneous jerkings of the injected paw. B. Bar-graphs of the effects of AppCH₂ppA (a, b) and AppNHppA (c, d) on the integral number of spontaneous jerkings. Measurements were made during the acute phase (0-6 min) and tonic phase (7-45 min) of the formalin response. C. Dose-response curves for AppCH₂ppA (a) and AppNHppA (b) during tonic and acute phases.

FIG. 7. Control injection of 100 μM (100 μL) of AppCH₂ppA and AppNHppA. Lack of nociceptive behavior. A. Time-course of the effects of subcutaneous AppCH₂ppA and AppNHppA on the number of the spontaneous jerkings of the injected paw. B. Bar-graphs of the effects of AppCH₂ppA, AppNHppA and saline on the integral number of spontaneous jerkings.

FIG. 8. Subcutaneous Ap₄A analogues reduced thermal hyperalgesia induced by complete Freund adjuvant (CFA). A. Time-course of the effects of 1 and 20 μM (100 μL) of AppCH₂ppA and AppNHppA on the paw withdrawal latency (PWL). B. Bar-graphs of the effects of AppCH₂ppA and AppNHppA on the integral PWL.

FIG. 9. Subcutaneous administration of AppCH₂ppA (50 μM, 100 μL) reduced thermal hyperalgesia in rats with Partial Sciatic Nerve Ligation (PSNL). Time-course of the effects of 50 μM (100 μL) of AppCH₂ppA on PWL.

FIG. 10. Intrathecal administration of AppCH₂ppA (20 μM, 100 μL) induced less pronounced analgesia, compared to intraplantar injection. A. Time-course of the effects of i.t. AppCH₂ppA on complete Freund adjuvant-induced thermal hyperalgesia (Hargreaves plantar test). B. Bar-graphs of the effects of i.t. AppCH₂ppA on rat PWL.

### DETAILED DESCRIPTION OF THE INVENTION

The invention uses dinucleoside polyphosphates, a family of compounds comprising two nucleoside moieties linked by a polyphosphate bridge. They can be represented by NpₙN, wherein N represents a nucleoside moiety, p represents a phosphate group and n is the number of phosphate groups (e.g. 2 to 7). Analogues of dinucleoside polyphosphates are compounds (typically synthetic) having a structure based on that of a dinucleoside polyphosphate, wherein one or more parts of the structure have been altered. For example the nucleobase, the sugar and/or the phosphate backbone may be modified, or partially or fully replaced, by another suitable moiety.

For example, one or more polyphosphate chain oxo-bridges may be replaced by a different bridge to increase the biological half-life of the compound in vivo. Such analogues may be designed to provide stability and/or biocompatibility. To achieve this, the analogue should be resistant to decomposition by biological systems in vivo. For example, the analogue may have increased hydrolytic stability, i.e. resistance to the breakdown of the molecule by specific enzyme cleavage (e.g. by one or more types of nucleotidase) and/or non-specific hydrolysis.

Preferably the compounds are diadenosine polyphosphates (e.g. of the type ApₙAs; where n is 2-7), such as naturally occurring purinergic ligands consisting of two adenosine moieties bridged by a chain of two or more phosphate residues attached at the 5′-position of each ribose ring. In particular, P¹, P⁴-diadenosine tetraphosphate (Ap₄A) and P¹, P⁵-diadenosine pentaphosphate (Ap₅A) are contemplated. These are present in high concentrations endogenously in the secretory granules of chromaffin cells and in rat brain synaptic terminals Upon depolarization, ApₙAs are released in a Ca²⁺-dependent manner and their potential role as neurotransmitters has been proposed. However, in spite of being well known for many years, pure functions of ApₙAs have been difficult to define because of both specific enzymatic cleavage and nonspecific hydrolytic breakdown. ApₙA analogues can be more stable than naturally occurring diadenosine polyphosphates with respect to both specific enzymatic and nonspecific hydrolytic breakdown.

### Preferred Compounds

Preferably, the dinucleoside polyphosphate (of the NPₙN type) for use in the present invention is a compound of formula (I):

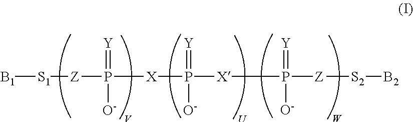

or a pharmaceutically acceptable salt thereof,
wherein X, X′ and Z are independently selected from

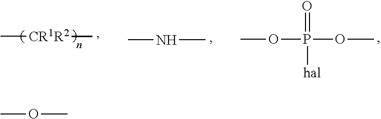

wherein R¹ and R² are independently selected from hydrogen, halogen, hydroxyl, cyano or an unsubstituted group selected from C₁₋₃ haloalkyl, C₁₋₃ alkyl, C₁₋₄ aminoalkyl and C₁₋₄ hydroxyalkyl, and n is selected from 1, 2, 3, 4, 5 and 6;
each Y is independently selected from ═S and ═O;
B₁ and B₂ are independently selected from a 5- to 7-membered carbon-nitrogen heteroaryl group which may be unfused or fused to a further 5- to 7-membered carbon-nitrogen heteroaryl group
S₁ and S₂ are independently selected from a bond, C₁₋₆ alkylene, C₂₋₆ alkenylene, C₂₋₆ alkynylene and a moiety of formula (II):

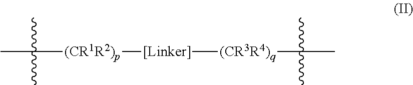

wherein
R¹, R², R³ and R⁴ independently represent hydrogen, halogen, hydroxyl, cyano or an unsubstituted group selected from C₁₋₃ haloalkyl, C₁₋₃ alkyl, C₁₋₄ aminoalkyl and C₁₋₄ hydroxyalkyl;
p and q independently represent 0, 1, 2 or 3, preferably 0, 1 or 2; and
[Linker] represents:
   (i) —O—, —S—, —C═O— or NH—;
   (ii) C₁₋₄ alkylene, C₂₋₄ alkenylene or C₂₋₄ alkynylene, which may optionally contain or terminate in an ether (—O—), thioether (—S—), carbonyl (—C═O—) or amino (—NH—) link, and which are optionally substituted with one or more groups selected from hydrogen, hydroxyl, halogen, cyano, —NR⁵R⁶ or an unsubstituted group selected from C₁₋₄ alkyl, C₂₋₄ alkenyl, C₁₋₄ alkoxy, C₂₋₄ alkenyloxy, C₁₋₄ haloalkyl, C₂₋₄ haloalkenyl, C₁₋₄ aminoalkyl, C₁₋₄ hydroxyalkyl, C₁₋₄ acyl and C₁₋₄ alkyl-NR⁵R⁶ groups, wherein R⁵ and R⁶ are the same or different and represent hydrogen or unsubstituted C₁₋₂ alkyl; or
   (iii) a 5 to 7 membered heterocyclyl, carbocyclyl or aryl group, which may be optionally substituted with one or more groups selected from hydrogen, hydroxyl, halogen, cyano, NR⁵R⁶ or an unsubstituted group selected from C₁₋₄ alkyl, C₂₋₄ alkenyl, C₁₋₄ alkoxy, C₂₋₄ alkenyloxy, C₁₋₄ haloalkyl, C₂₋₄ haloalkenyl, C₁₋₄ aminoalkyl, C₁₋₄ hydroxyalkyl, C₁₋₄ acyl and C₁₋₄ alkyl-NR⁵R⁶ groups, wherein R⁵ and R⁶ are the same or different and represent hydrogen or unsubstituted C₁₋₂ alkyl;
      V is selected from 0, 1, 2, 3, 4 and 5;
      U is selected from 0, 1, 2, 3, 4 and 5;
      W is selected from 0, 1, 2, 3, 4 and 5; and
      V plus U plus W is an integer from 2 to 7.

As used herein, a C₁₋₄ alkyl group or moiety is a linear or branched alkyl group or moiety containing from 1 to 4 carbon atoms. Examples of C₁₋₄ alkyl groups include methyl, ethyl, n-propyl, i-propyl, n-butyl, i-butyl and t-butyl.

As used herein, a C₂₋₄ alkenyl group or moiety is a linear or branched alkenyl group or moiety having at least one double bond of either E or Z stereochemistry where applicable and containing from 2 to 4 carbon atoms, such as —CH═CH₂ or —CH₂—CH═CH₂, —CH₂—CH₂—CH═CH₂, —CH₂—CH═CH—CH₃, —CH═C(CH₃)—CH₃ and —CH₂—C(CH₃)═CH₂.

As used herein, a C₁₋₆ alkylene group or moiety is a linear or branched alkylene group or moiety, for example a C₁₋₄ alkylene group or moiety. Examples include methylene, n-ethylene, n-propylene and —C(CH₃)₂— groups and moieties.

As used herein, a C₂₋₆ alkenylene group or moiety is a linear or branched alkenylene group or moiety, for example a C₂₋₄ alkenylene group or moiety. Examples include —CH═CH—, —CH═CH—CH₂—, —CH₂—CH═CH— and —CH═CH—CH═CH—.

As used herein, a C₂₋₆ alkynylene group or moiety is a linear or branched alkynylene group or moiety, for example a C₂₋₄ alkynylene group or moiety. Examples include and —CH₂—C≡C—.

As used herein, a halogen atom is chlorine, fluorine, bromine or iodine.

As used herein, a C₁₋₄ alkoxy group or C₂₋₄ alkenyloxy group is typically a said C₁₋₄ alkyl group or a said C₂₋₄ alkenyl group respectively which is attached to an oxygen atom.

A haloalkyl or haloalkenyl group is typically a said alkyl or alkenyl group respectively which is substituted by one or more said halogen atoms. Typically, it is substituted by 1, 2 or 3 said halogen atoms. Preferred haloalkyl groups include perhaloalkyl groups such as —CX₃ wherein X is a said halogen atom, for example chlorine or fluorine.

Preferably, a C₁₋₄ or C₁₋₃ haloalkyl group as used herein is a C₁₋₃ fluoroalkyl or C₁₋₃ chloroalkyl group, more preferably a C₁₋₃ fluoroalkyl group.

As used herein, a C₁₋₄ aminoalkyl group is a C₁₋₄ alkyl group substituted by one or more amino groups. Typically, it is substituted by one, two or three amino groups. Preferably, it is substituted by a single amino group.

As used herein, a C₁₋₄ hydroxyalkyl group is a C₁₋₄ alkyl group substituted by one or more hydroxy groups. Typically, it is substituted by one, two or three hydroxy groups. Preferably, it is substituted by a single hydroxy group.

As used herein, a C₁₋₄ acyl group is a group C(═O)R, wherein R is a said C₁₋₄ alkyl group.

As used herein, a 5 to 7 membered heterocyclyl group includes heteroaryl groups, and in its non-aromatic meaning relates to a saturated or unsaturated non-aromatic moiety having 5, 6 or 7 ring atoms and containing one or more, for example 1 or 2, heteroatoms selected from S, N and O, preferably O. Illustrative of such moieties are tetrahydrofuranyl and tetrahydropyranyl. For example, the heterocyclic ring may be a furanose or pyranose ring.

As used herein, a 5- to 7-membered carbon-nitrogen heteroaryl group is a monocyclic 5- to 7-membered aromatic ring, such as a 5- or 6-membered ring, containing at least one nitrogen atom, for example 1, 2, 3 or 4 nitrogen atoms. The 5- to 7-membered carbon-nitrogen heteroaryl group may be fused to another 5- to 7-membered carbon-nitrogen heteroaryl group.

As used herein, a 5 to 7 membered carbocyclyl group is a non-aromatic, saturated or unsaturated hydrocarbon ring having from 5 to 7 carbon atoms. Preferably it is a saturated or mono-unsaturated hydrocarbon ring (i.e. a cycloalkyl moiety or a cycloalkenyl moiety) having from 5 to 7 carbon atoms. Examples include cyclopentyl, cyclohexyl, cyclopentenyl and cyclohexenyl.

As used herein, a 5 to 7 membered aryl group is a monocyclic, 5- to 7-membered aromatic hydrocarbon ring having from 5 to 7 carbon atoms, for example phenyl.

In one aspect X and X′ are independently —NH—.

In one aspect X and X′ are independently

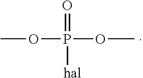

In one aspect X and X′ are independently

—(CR¹R²)ₙ—
wherein at least one of R¹ and R² is H, Cl, Br or F.

Preferably both R¹ and R² are H.

Preferably n is 1, 2 or 3, preferably 1 or 2.

Preferably at least one of X and X′ is not —O—, i.e. not all X and X′ are —O—.

Preferably X and X′ are independently selected from NH and

—(CR¹R²)ₙ—
wherein R¹ and R² are both H and n is 1 or 2.

In one aspect at least one Y is ═S.

In one aspect each Y group is ═S.

In one aspect at least one Y is ═O.

Preferably each Y group is ═O.

In one aspect at least one Z is

—(CR¹R²)ₙ—

In one aspect each Z is

—(CR¹R²)ₙ—
wherein at least one of R¹ and R² is H, Cl, Br or F.

Preferably both R¹ and R² are H. Thus, in one aspect Z is

—(CR¹R²)ₙ—
and R¹ and R² are both H.

Preferably n is 1, 2 or 3, preferably 1 or 2.

In one aspect at least one Z is —NH—.

In one aspect each Z is —NH—.

In one aspect at least one Z is —O—.

Preferably each Z is —O—.

B₁ and B₂ are preferably independently selected from purine and pyrimidine nucleic acid bases, preferably adenine, guanine, thymine, cytosine, uracil, hypoxanthine, xanthine, 1-methyladenine, 7-methylguanine, 2-N,N-dimethylguanine, 5-methylcytosine or 5,6-dihydrouracil. Uracil may be attached to S₁ or S₂ via N (i.e. uridine structure) or C (i.e. pseudouridine structure).

Preferably, B₁ and B₂ are independently selected from adenine, guanine, and uracil. Preferably at least one of B₁ and B₂ is adenine.

Thus, for example, at least one of B₁ and B₂ may be adenine and the other of B₁ and B₂ may be guanine, or at least one of B₁ and B₂ may be adenine and the other of B₁ and B₂ may be uracil.

Preferably, B₁ and B₂ are both adenine, or one of B₁ and B₂ is adenine and the other is guanine.

S₁ and S₂ are preferably independently selected from a bond, C₁₋₆ alkylene, C₂₋₆ alkenylene, C₂₋₆ alkynylene and a moiety of formula (III) or (IV):

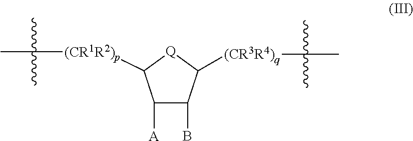

wherein
R¹, R², R³ and R⁴ independently represent hydrogen, halogen, hydroxyl, cyano or an unsubstituted group selected from C₁₋₃ haloalkyl, C₁₋₃ alkyl, C₁₋₄ aminoalkyl and C₁₋₄ hydroxyalkyl;
p and q independently represent 0 or 1;
Q represents —O—, —S—, —C═O—, NH— or CH₂; and
A and B independently represent hydrogen, hydroxyl, halogen, or an unsubstituted group selected from C₁₋₄ alkoxy, C₁₋₄ aminoalkyl, C₁₋₄ hydroxyalkyl, C₁₋₄ acyl and NR⁵R⁶ groups, wherein R⁵ and R⁶ are the same or different and represent hydrogen or unsubstituted C₁₋₂ alkyl;

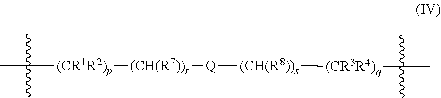

wherein
R¹, R², R³ and R⁴ independently represent hydrogen, halogen, cyano or an unsubstituted group selected from C₁₋₃ haloalkyl, C₁₋₃ alkyl, C₁₋₄ aminoalkyl and C₁₋₄ hydroxyalkyl;
Q represents —O—, —S—, —C═O—, NH— or CH₂; and
R⁷ and R⁸ independently represent hydrogen, hydroxyl, halogen, cyano, NR⁵R⁶ or an unsubstituted group selected from C₁₋₄ alkyl, C₂₋₄ alkenyl, C₁₋₄ alkoxy, C₂₋₄ alkenyloxy, C₁₋₄ haloalkyl, C₂₋₄ haloalkenyl, C₁₋₄ aminoalkyl, C₁₋₄ hydroxyalkyl, C₁₋₄ acyl and C₁₋₄ alkyl-NR⁵R⁶ groups, wherein R⁵ and R⁶ are the same or different and represent hydrogen or unsubstituted C₁₋₂ alkyl; and
p, q, r and s independently represent 0 or 1.

S₁ and S₂ are preferably independently selected from a moiety of formula (III) or (IV) as set out above, in which preferably:
R¹, R², R³ and R⁴ independently represent hydrogen, fluoro, chloro, or unsubstituted C₁₋₃ alkyl; more preferably hydrogen;
Q represents —O—;
A and B independently represent hydrogen, hydroxyl, fluoro, chloro, methoxy, formyl or NH₂, more preferably hydrogen or hydroxyl; and
R⁷ and R⁸ independently represent hydrogen, hydroxyl, fluoro, chloro, or an unsubstituted group selected from C₁₋₄ alkyl, C₁₋₄ haloalkyl, C₁₋₄ hydroxyalkyl and C₁₋₄ alkyl-NH₂, more preferably hydrogen, hydroxyl or unsubstituted methyl, ethyl, —CH₂OH or —CH₂CH₂OH.

S₁ and S₂ may preferably be independently selected from D-ribofuranose, 2′-deoxy-D-ribofuranose, 3′-deoxy-D-ribofuranose, L-arabinofuranose (corresponding to moieties of formula (III)), and ring opened forms thereof (corresponding to moieties of formula (IV)).

In one preferred embodiment, at least one of S₁ and S₂ is D-ribofuranose, i.e. a moiety of formula (III)) in which R¹ and R² are hydrogen, p is 1, q is 0, Q is —O— and A and B are hydroxyl:

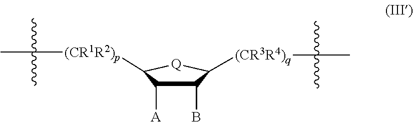

When S₁ and/or S₂ is a ring opened form, the ring opening is preferably between the 2′ and 3′ positions of the D-ribofuranose, 2′-deoxy-D-ribofuranose, 3′-deoxy-D-ribofuranose or L-arabinofuranose ring.

In one preferred embodiment, at least one of S₁ and S₂ is a ring opened form of D-ribofuranose, for example a moiety of formula (IV) in which R¹ and R² are hydrogen, p is 1, q is 0, Q is —O—, r is 1, s is 1 and R⁷ and R⁸ are each —CH₂OH.

Preferably S₁ and S₂ are the same. Thus preferably, S₁ and S₂ are both D-ribofuranose or both a ring opened form of D-ribofuranose as described above.

The sum of V, U and W may be 2, 3, 4, 5, 6 or 7.

Preferably V plus U plus W is 4 or 5.

Preferably U is 0, 1 or 2.

Preferably V is 2.

Preferably W is 2.

In a preferred embodiment, U is 0. Thus the dinucleoside polyphosphate for use in the present invention is preferably a compound of formula (I′):

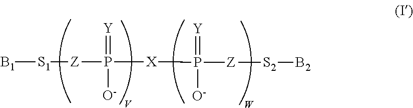

wherein all symbols are as defined above, X is not —O— and V plus W is a integer from 2 to 7.

Thus, the sum of V and W in formula (I′) may be 2, 3, 4, 5, 6 or 7. Preferably V plus W is 4 or 5. Preferably V is 2 and/or W is 2.

In a preferred embodiment, each Y is ═O and each Z is —O—.

In a more preferred embodiment, each Y is ═O and each Z is —O—, and both S₁ and S₂ are a moiety of formula (III) or (IV) as set out above. Preferably, both S₁ and S₂ are the same and are both D-ribofuranose or both a ring opened form of D-ribofuranose. Thus the dinucleoside polyphosphate analogue of the present invention is preferably a compound of formula (IA) or (IB):

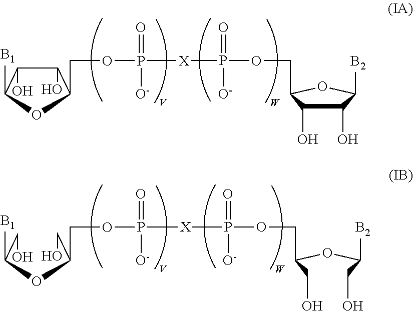

Preferably, the dinucleoside polyphosphate analogue of the present invention is a compound of formula (IA) or (IB) wherein V plus W is 4 or 5. More preferably, the dinucleoside polyphosphate analogue of the present invention is a compound of formula (IA) or (IB) wherein at least one of B₁ and B₂ is adenine, or one of B₁ and B₂ is adenine and the other is guanine.

Thus, in a more preferred embodiment, each Y is ═O and each Z is —O—, both S₁ and S₂ are the same and are both D-ribofuranose or both a ring opened form of D-ribofuranose, and B₁ and B₂ are both adenine, or one of B₁ and B₂ is adenine and the other is guanine. Thus the dinucleoside polyphosphate analogue of the present invention is preferably a dinucleoside polyphosphate compound of formula (IC) to (IF):

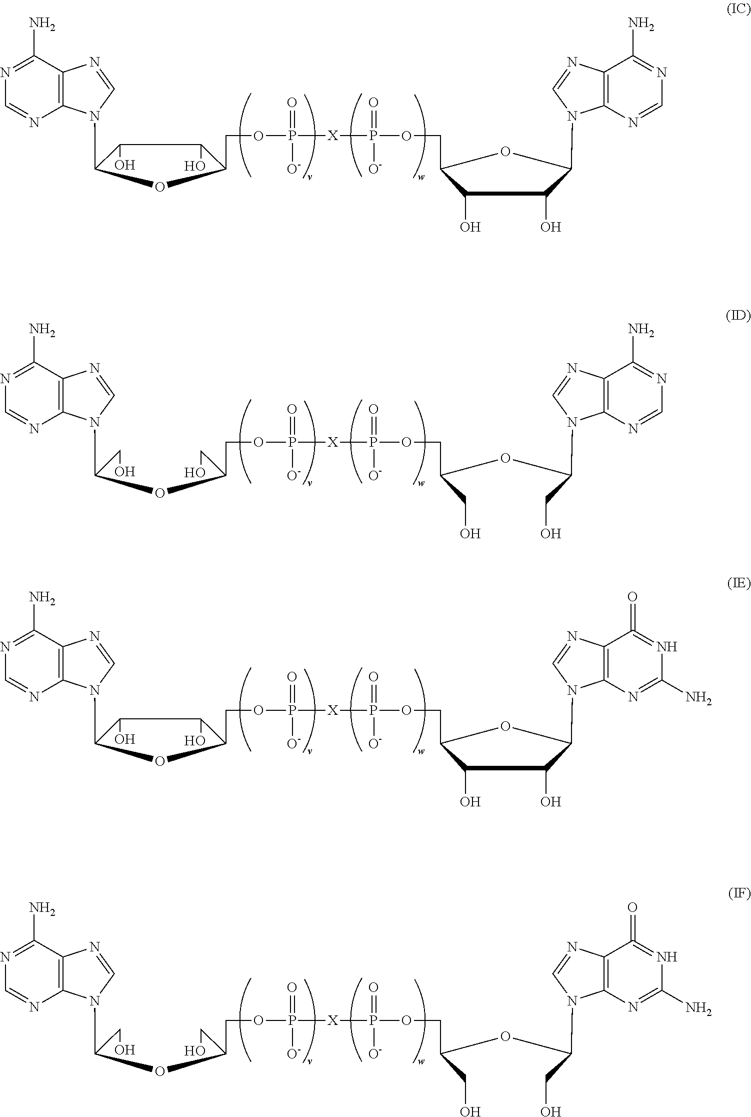

Preferably, the dinucleoside polyphosphate analogue is a compound of formula (IC) to (IF) wherein V plus W is 4 or 5. Thus, in a preferred aspect of the invention, the dinucleoside polyphosphate analogue is chosen among the group consisting of Ap₄A analogues, Ap₅A analogues, Ap₄G analogues and Ap₅G analogues.

In a preferred embodiment, V and W are the same. Thus in the above compounds of formula (I′) and (IA) to (IF), V and W are preferably each 2. In a further preferred embodiment, the dinucleoside polyphosphate analogue is symmetrical.

In a preferred aspect of the invention, the dinucleoside polyphosphate analogue is chosen among the group consisting of AppCH₂ppA, AppNHppA, A_{diol}ppCH₂ppA_{diol}, A_{diol}ppNHppA_{diol}, AppCH₂ppG, AppNHppG, A_{diol}ppCH₂ppG_{diol} and A_{diol}ppNHppG_{diol}:

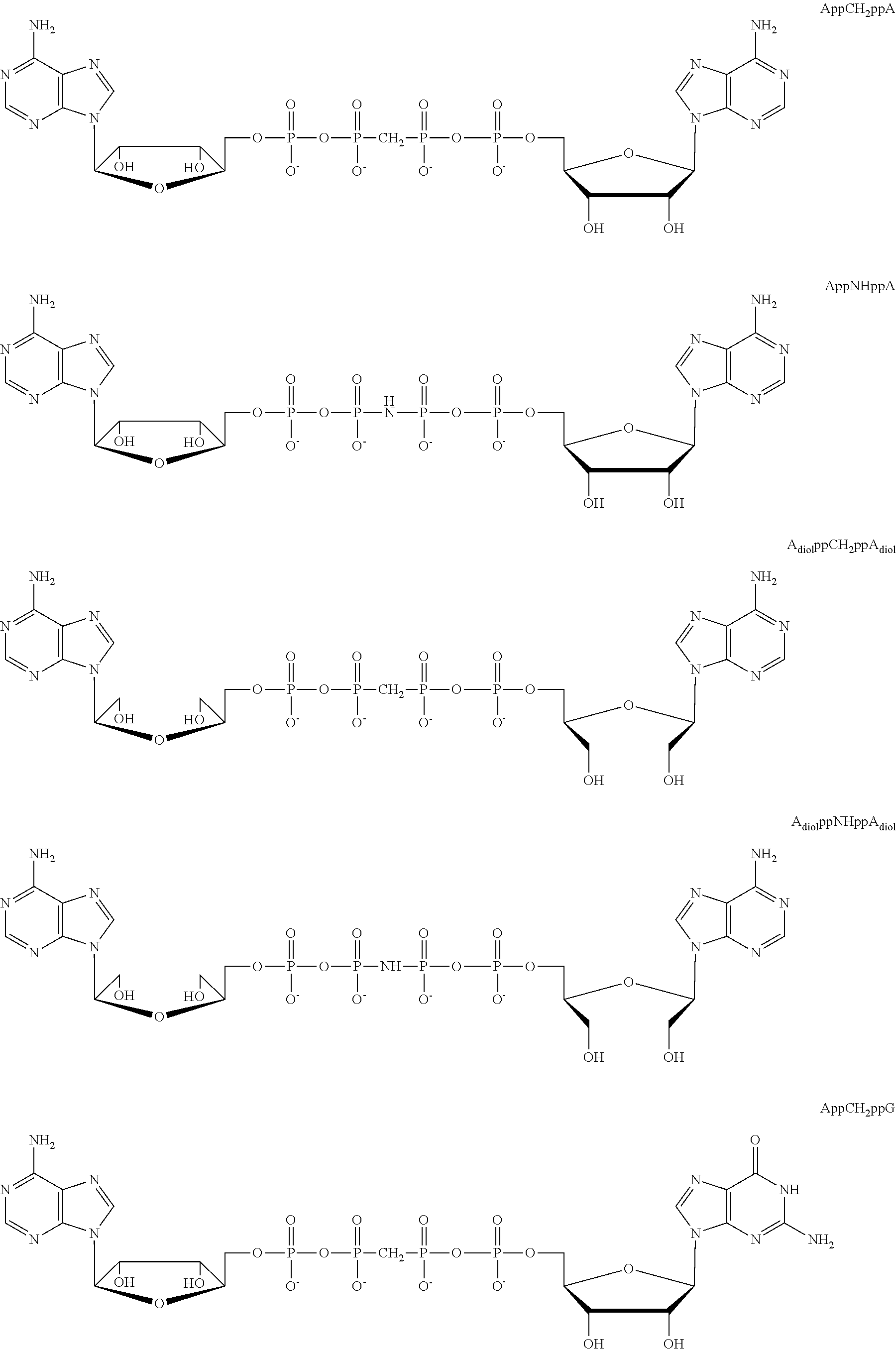

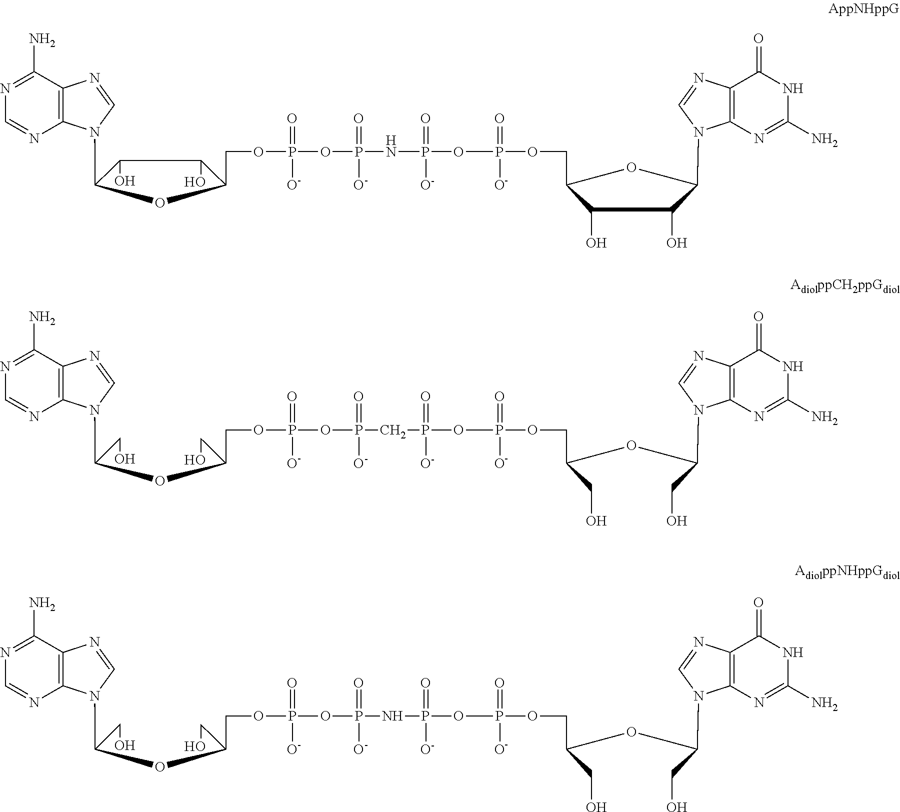

As demonstrated in the Examples of the present application, such dinucleoside polyphosphate analogues potently inhibit or down-regulate P2X3 receptors via enhancement of desensitization and exert potent antinociceptive activities on an in vivo animal model of inflammatory pain.

Dinucleoside polyphosphates of general formula (I) and their preparation are disclosed in WO 2006/082397.

However, WO 2006/082397 does not disclose the binding properties of dinucleoside polyphosphate analogues on P2X3 receptors, and in particular the inhibitor and partial (or super-) agonist properties of dinucleoside polyphosphate analogues on P2X3 receptors. WO 2006/082397 does not disclose their specific action on chronic hyperalgesia when compared to acute hyperalgesia. Also, although WO 2006/082397 discloses an effect of AppCH₂ppA on the central nervous system, via a hippocampal preparation, it does not disclose a specific effect of dinucleoside polyphosphate analogues on the peripheral nervous system.

### Mechanism

The compound of the invention can act (e.g. solely) on the P2X3 receptor, and appears to be selective for that (P2X3) receptor (only). In other words, the compound may to act only on, or be selective for only, the P2X3 receptor (when considering the P2X receptor family). Suitably the compound does not bind to, is not selective for, and/or does not act on either the P2X4 and/or the P2X7 receptors. Indeed, the compound similarly appears not to act on, or bind, the P2X2 receptor either.

The compound may not act as an antagonist. Instead, it can act as an agonist, or super-agonist. It is believed that the compound acts on the relevant receptor(s) via or using a high affinity desensitisation (HAD) method. The compound is thought to bind to the receptor, but do not detach or fall away from the receptor particularly quickly. It is thought that the mechanism of action thus means that the receptor fires once, and may then be blocked, such as for a significant period of time. This is therefore different from the prior art findings, and prior art (pain inhibiting) compounds, which exhibit a typical receptor antagonism inhibition.

The compound thus appears to act via a high affinity desensitisation (HAD) inhibition mechanism. This appears to be a new mechanism, in particular for those compounds that may be able to inhibit (and therefore be used in the treatment of) pain. Therefore, the invention additionally relates to a compound that can act via the HAD mechanism, or involve the HAD mechanism (at one or more receptors). The pain inhibitory compounds therefore used in the invention are thought to be the first in their class, and act via an entirely new mechanism, which allows them to be distinguished over prior art pain inhibitory methods. Note that the HAD mechanism is detailed in Example 2.

The compound of the invention are thought to have a high degree of affinity for P2X3, perhaps more so than other similar compounds in the prior art. Since they appear not to act on either the P2X4 and/or P2X7 receptors, or even the P2X2 receptor either, this makes the compounds of the invention particularly suitable for treating pain. Note that the compounds and the treatment is not CNS related, and can be used in local treatment.

The inventors have now shown that different dinucleoside polyphosphate analogues are able to potently inhibit P2X3 receptors in vitro. They have also shown that different dinucleoside polyphosphate analogues are able to exert antinociceptive activities on moderate to chronic pain and to act on the peripheral nervous system, based on different in vivo animal models of inflammatory pain. These data are described in greater detail in the “Examples” section of the present application, in particular Example 2 which details the HAD mechanism

The group of receptors containing P2X3 subunits comprise homomeric P2X3 receptors and heteromeric P2X23 receptors, and are designated as “P2X3 receptors” in the present application. Preferably, the dinucleoside polyphosphate analogues of the present invention target P2X3 receptors, and show little or no activity in relation to other P2X receptors, such as homomeric P2X2 receptors, and/or P2X4 or P2X7 receptors.

As demonstrated in the Examples of the present application, stable dinucleoside polyphosphate analogues inhibited pain transducing ATP-gated P2X3 receptors when administered at concentrations below the threshold needed to elicit macroscopic receptor-mediated currents that induce pain sensations. Without wishing to be bound by theory, these results suggest that these analogues mediate effects by inducing stabilization of the desensitized receptor state, and act via the HAD inhibition mechanism. Results of in vitro tests correlated with the ability of these two stable dinucleoside polyphosphate analogues to reduce inflammatory pain in behavioural tests.

In a preferred aspect, the dinucleoside polyphosphate analogue for use as described herein is a partial or super-agonist of a P2X3 receptor. The partial agonist activity exhibited by the analogue may be, for example, a lower activity than a known full P2X3 agonist such as α,β-methylene-ATP (e.g. less than 50% activity of the activity of the know full P2X3 agonist).

In particular, the dinucleoside polyphosphate analogue of the present invention may show partial (or super-)agonist activity of a P2X3 receptor at higher concentrations and antagonistic (or inhibitory) activity at lower concentrations. Thus for example the analogue may show partial agonist activity at concentrations up to saturating concentration. On the other hand, the analogue may show inhibitory activity of a P2X3 receptor at lower concentrations, e.g. when administered at concentrations below the threshold needed to elicit macroscopic receptor-mediated currents that induce pain sensations (for example when administered in the amounts as described herein).

Wildman et al (1999) and McDonald et al (2002) teach that diadenosine polyphosphate might be full agonists of P2X3 receptors. These documents do not disclose the partial agonist properties of NpₙN analogues on P2X3 receptors, as shown in the present invention.

The main requirements of potential analgesics operating via promotion of desensitization are to reduce agonist activity and induce stable effects. The present inventors prepared several stable NpₙN analogues and demonstrated that they are only partial agonists of native and recombinant P2X3 receptors. By contrast, as noted above, they efficiently inhibited P2X3 receptors when administered at concentrations below the threshold needed to elicit macroscopic receptor-mediated currents that induce pain sensations. This inhibitory action was almost absent in nodose ganglia neurons, where ATP or α,β-meATP agonists are able to generate mainly slow type currents mediated by homomeric P2X2 or heteromeric P2X2P2X3 receptors. In contrast, inhibition was strong in trigeminal ganglia neurons that preferentially express the homomeric P2X3 receptors subtype. Hence in this experiment the NpₙN analogues appeared to mediate selective inhibitory effects on homomeric P2X3 subunit containing receptors.

In view of the effects described above, the dinucleoside polyphosphate analogues of the present invention are preferably for use in the treatment of pain. For example, the dinucleoside polyphosphate analogue may be administered in association with a pharmaceutically acceptable vehicle in order to treat pain in the human or animal body.

In particular, the dinucleoside polyphosphate analogues of the present invention may be for use in the inhibition of a homomeric P2X3 receptor. Thus, the analogues of the present invention may be for use in the treatment of pain by inhibition of a homomeric P2X3 receptor.

The present invention also relates to a method of inhibition (including prevention and/or reduction) of pain, suitably via a transducing ATP-gated P2X3 receptor (preferably a homomeric P2X3 receptor) comprising administering an effective amount of a dinucleoside polyphosphate analogue (as described herein) or a pharmaceutically acceptable salt thereof, and to use of a dinucleoside polyphosphate analogue (as described herein) or a pharmaceutically acceptable salt thereof, in the manufacture of a medicament for inhibition of a pain transducing ATP-gated P2X3 receptor (preferably a homomeric P2X3 receptor). The invention also relates to a method of treating pain, comprising administering an effective amount of a dinucleoside polyphosphate analogue (as described herein) or a pharmaceutically acceptable salt thereof, and to use of a dinucleoside polyphosphate analogue (as described herein) or a pharmaceutically acceptable salt thereof, in the manufacture of a medicament for the treatment of pain.

### Pain Types and Therapies

Pain may be classified into different types. Nociceptive pain is mediated by pain receptors in response to injury, disease or inflammation. Neuropathic pain is a neurological disorder caused by damage to the pain transmission system from periphery to brain. Psychogenic pain is pain associated with actual mental disorder.

Pain may be chronic or acute, depending on its duration. Chronic pain can generally be described as pain that has lasted for a long time, for example beyond the expected period of healing. Typically, chronic pain is pain which lasts for 3 months or more. Pain which lasts for less than 30 days can be classed as acute pain, and pain of intermediate duration can be described as moderate or subacute pain.

The pain treated by the present invention may be associated with, for example, symptoms associated with one or more of inflammation (for example from cancer, arthritis or trauma), back pain (including sciatic back pain), trapped nerve, arthritic pain, cancer-related pain, dental pain, endometriosis, birthing-related pain (e.g. pre- and/or post-partum), post-surgical pain or trauma.

As described above, the dinucleoside polyphosphate analogues as described herein are particularly active against P2X3 receptors (especially homomeric P2X3 receptors). They can therefore be administered in low amounts compared with known agents for the treatment of pain.

Thus for the treatment (including prevention and/or reduction) of pain, the dinucleoside polyphosphate analogue is preferably administered in an amount of about 0.01 to 1000 nmolkg, preferably from 0.1 to 500 nmolkg, for example from 0.01 to 500 μg/kg, preferably from 0.1 to 250 μg/kg. In one embodiment, the dinucleoside polyphosphate analogue is preferably administered in an amount of from 0.01 to 10 μg/kg, preferably 0.05 to 5 μg/kg, more preferably from 0.1 to 2 μg/kg.

In one preferred embodiment of the present invention, the dinucleoside polyphosphate analogues are for use in treatment of moderate to chronic pain. The moderate to chronic pain may be mediated by nociceptive and/or neuropathic mechanisms. Preferably, the moderate to chronic pain may be nociceptive, for example, associated with at least one of the symptoms chosen among the group consisting of: inflammation (for example from cancer or arthritis), back pain, arthritic pain, cancer-related pain, dental pain, endometriosis and post-surgical pain. In particular, the moderate to chronic pain may be associated with inflammation, back pain, arthritis or cancer-related pain, particularly inflammation or cancer-related pain.

Thus, the present invention also relates to a dinucleoside polyphosphate analogue (as described herein) or a pharmaceutically acceptable salt thereof, for use in the treatment of moderate to chronic pain, in particular moderate to chronic neuropathic or moderate to chronic nociceptive pain, for example moderate to chronic nociceptive pain associated with at least one of the symptoms chosen among the group consisting of inflammation (for example from cancer or arthritis), back pain, arthritic pain, cancer-related pain, dental pain, endometriosis and post-surgical pain. In particular, the moderate to chronic pain may be associated with inflammation, back pain, arthritis or cancer-related pain, particularly inflammation or cancer-related pain.

The present invention also relates to a method of treating moderate to chronic pain, comprising administering an effective amount of a dinucleoside polyphosphate analogue (as described herein) or a pharmaceutically acceptable salt thereof, and to use of a dinucleoside polyphosphate analogue (as described herein) or a pharmaceutically acceptable salt thereof, in the manufacture of a medicament for the treatment of moderate to chronic pain. In particular, the moderate to chronic pain is moderate to chronic neuropathic or moderate to chronic nociceptive pain, for example moderate to chronic nociceptive pain associated with at least one of the symptoms chosen among the group consisting of inflammation (for example from cancer or arthritis), back pain, arthritic pain, cancer-related pain, dental pain, endometriosis and post-surgical pain. In particular, the moderate to chronic pain may be associated with inflammation, back pain, arthritis or cancer-related pain, particularly inflammation or cancer-related pain.

### Dosages

For the treatment of moderate to chronic pain, the dinucleoside polyphosphate analogue of the present invention is preferably administered in an amount of about 0.01 to 100 nmolkg, preferably from 0.1 to 10 nmolkg. Thus the compound may be administered in an amount of from 0.01 to 10 μg/kg, preferably 0.05 to 5 μg/kg, more preferably from 0.1 to 2 μg/kg.

Preferably, the dinucleoside polyphosphate analogue is one of the preferred analogues described above. In particular, the present invention relates to a dinucleoside polyphosphate analogue for use in the treatment of moderate to chronic pain, preferably wherein the dinucleoside polyphosphate analogue is chosen among the group consisting of: AppCH₂ppA, AppNHppA, A_{diol}ppCH₂ppA_{diol}, A_{diol}ppNHppA_{diol}, AppCH₂ppG, AppNHppG, A_{diol}ppCH₂ppG_{diol} and A_{diol}ppNHppG_{diol}.

When used for the treatment of moderate to chronic pain, the compound chosen among the group consisting of: AppCH₂ppA, AppNHppA, A_{diol}ppCH₂ppA_{diol}, A_{diol}ppNHppA_{diol}, AppCH₂ppG, AppNHppG, A_{diol}ppCH₂ppG_{diol} and A_{diol}ppNHppG_{diol} is preferably administered in association with a pharmaceutically acceptable vehicle, wherein the dose of compound administered to a subject in need of treatment is from about 0.01 to 100 nmolkg, preferably from 0.1 to 10 nmolkg. Thus the compound may be administered in an amount of from 0.01 to 10 μg/kg, preferably 0.05 to 5 μg/kg, more preferably from 0.1 to 2 μg/kg.

For example, for a typical human of about 70 kg, the amount of the compound administered may be between about 1 and about 100 nmol, more preferably between about 10 and about 100 nmol, and even more preferably between about 10 and about 50 nmol.

In another embodiment, the dinucleoside polyphosphate analogues of the present invention are for use in the treatment of acute pain or subacute pain. Thus the present invention also relates to a method of treating acute pain or subacute pain, comprising administering an effective amount of a dinucleoside polyphosphate analogue (as described herein) or a pharmaceutically acceptable salt thereof, and to use of a dinucleoside polyphosphate analogue (as described herein) or a pharmaceutically acceptable salt thereof, in the manufacture of a medicament for the treatment of acute pain or subacute pain.

The present invention also relates to a dinucleoside polyphosphate analogue (as described herein) or a pharmaceutically acceptable salt thereof, for use in the treatment of acute pain or subacute pain.

The acute pain or subacute pain may preferably be associated with post-surgical pain, dental pain, birthing-related pain, trauma or inflammation (for example resulting from trauma).

For the treatment of acute pain or subacute pain, the dinucleoside polyphosphate analogue of the present invention is preferably administered in an amount of about 50 to 1000 nmolkg, preferably from 50 to 500 nmolkg, more preferably from 75 to 300 nmolkg. Thus the compound may be administered in an amount of from about 10 to 500 μg/kg, preferably from 50 to 250 μg/kg.

Preferably, the dinucleoside polyphosphate analogue is one of the preferred analogues described above. In particular, the present invention relates to a dinucleoside polyphosphate analogue for use in the treatment of acute pain or subacute pain, preferably wherein the dinucleoside polyphosphate analogue is chosen among the group consisting of: AppCH₂ppA, AppNHppA, AppCH₂ppG, AppNHppG, A_{diol}ppCH₂ppG_{diol} and A_{diol}ppNHppG_{diol}, preferably administered in the amounts described above.

The dinucleoside polyphosphate analogues of the present invention may be administered in a variety of dosage forms. Thus, the dinucleoside polyphosphate analogues may be administered orally, for example as tablets, troches, lozenges, aqueous or oily suspensions, dispersible powders or granules. The dinucleoside polyphosphate analogues may also be administered parenterally, either subcutaneously, transdermally (by injection), intravenously, intramuscularly, intrasternally or by infusion techniques. The dinucleoside polyphosphate analogues may also be administered rectally, for example in the form of a suppository. A physician will be able to determine the required route of administration for each particular patient. Preferably, the dinucleoside polyphosphate analogues are administered by subcutaneous injection.

In another embodiment, the present invention relates to a composition comprising a dinucleoside polyphosphate analogue (as described herein) or a pharmaceutically acceptable salt thereof in an amount of from 0.01 to 3500 μg and a pharmaceutically acceptable excipient. The maximum amount of the dinucleoside polyphosphate analogue in the composition is preferably 1500 μg, preferably 1000 μg, more preferably 500 μg, particularly preferably 250 μg or 150 μg.

For administration to a human, based on a weight of 70 kg, the composition preferably comprises the dinucleoside polyphosphate analogue or a pharmaceutically acceptable salt thereof in an amount of from 0.5 to 3500 μg. Preferred maximum amounts are those described above. Expressed in nmol, the amount of the dinucleoside polyphosphate analogue in the composition may be from 0.5 to 5000 nmol, preferably from 1 to 2500 nmol, preferably 5 to 1000 nmol, more preferably 5 to 500 nmol, particularly preferably 10 to 100 nmol.

It would of course be necessary to recalibrate the above amounts for veterinary use, depending on animal body weight. For example, a typical composition for veterinary use may comprise the dinucleoside polyphosphate analogue or a pharmaceutically acceptable salt thereof in an amount of from 0.01 to 200 μg, preferably from 0.05 to 100 μg

Preferably, the dinucleoside polyphosphate analogue in the composition is one of the preferred analogues described above, in particular AppCH₂ppA, AppNHppA, A_{diol}ppCH₂ppA_{diol}, A_{diol}ppNHppA_{diol}, AppCH₂ppG, AppNHppG, A_{diol}ppCH₂ppG_{diol}; or A_{diol}ppNHppG_{diol}.

### Compositions

Preferably, the composition is formulated for subcutaneous injection.

The formulation of the dinucleoside polyphosphate analogues will depend upon factors such as the nature of the exact agent, whether a pharmaceutical or veterinary use is intended, etc. An agent for use in the present invention may be formulated for simultaneous, separate or sequential use.

The dinucleoside polyphosphate analogues are typically formulated for administration in the present invention with a pharmaceutically acceptable excipient (such as a carrier or diluents). The pharmaceutical carrier or diluent may be, for example, an isotonic solution. For example, solid oral forms may contain, together with the active compound, diluents, e.g. lactose, dextrose, saccharose, cellulose, corn or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tableting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Formulations for oral administration may be formulated as controlled release formulations, for example they may be formulated for controlled release in the large bowel.

Solutions for intravenous administration or infusion may contain as carrier, for example, sterile water or preferably they may be in the form of sterile, aqueous, isotonic saline solutions.

The dose of the dinucleoside polyphosphate analogues may be determined according to various parameters, especially according to the substance used; the age, weight and condition of the patient to be treated; the route of administration; and the required regimen.

Again, a physician will be able to determine the required route of administration and dosage for any particular patient. A typical daily dose is from about 0.01 to 1000 μg per kg of body weight, according to the age, weight and conditions of the individual to be treated, the type and severity of the condition (e.g. of the pain) and the frequency and route of administration. Daily dosage levels may be, for example, from 0.01 to 500 μg/kg. In the treatment of moderate to chronic pain, suitable daily dosage levels may be from about 0.01 to 20 μg/kg, preferably from 0.05 to 15 μg/kg, preferably from 0.1 to 10 μg/kg. In the treatment of acute pain or subacute pain, suitable daily dosage levels may be from about 10 to 1000 μg/kg, preferably from 50 to 500 μg/kg.

The dinucleoside polyphosphate analogues as described herein may be administered alone or in combination. They may also be administered in combination with another pharmacologically active agent, such as another agent for the treatment of pain, for example an opioid, non-opioid or NSAID. For example, the dinucleoside polyphosphate analogues for use according to the present invention may be combined with an opioid such as oxycodone (for example OxyContin®; controlled-release oxycodone HCl; Purdue Pharma L.P.). The combination of agents may be may be formulated for simultaneous, separate or sequential use.

All publications and patent applications mentioned in this specification are indicative of the level of those skilled in the art to which this invention pertains. All publications and patent applications are herein incorporated by reference to the same extent as if each individual publication or patent application was specifically and individually to be incorporated by reference.

Although the foregoing invention has been described in some detail by way of illustration and example for purposes of understanding, it will be clear to those skilled in the art that certain changes and modifications may be practiced within the scope of the appended claims.

The following Examples illustrate the invention:

### EXAMPLES

### Ap₄a Analogue Synthesis

AppNHppA and AppCH₂ppA were prepared using a development of the LysU-mediated biosynthetic process described previously (Melnik et al., 2006, WO 2006/0823297), with rigorous purification by HPLC (Wright et al., 2003, 2004 and 2006).

### Cell Cultures and Transfection

Rat trigeminal (TG), nodose (NG) or dorsal root ganglion (DRG) neurons in culture were prepared as described previously (Sokolova et al., 2001; Simonetti et al., 2006). Neurons were plated on poly-L-lysine (0.2 mg/mL)-coated Petri dishes and cultured for 1 to 2 days under an atmosphere containing 5% CO₂. Cells were used within 2 days of plating when they lacked processes. HEK293T cells were prepared as reported previously (Fabbretti et al., 2004; Sokolova et al., 2004) and transfected with rat full-length P2X3 cDNA subcloned into pIRES2-EGFP (Clontech, Mountain View, Calif., USA).

### Electrophysiological Recordings

Trigeminal, nodose, DRG neurons or HEK cells were recorded in the whole-cell configuration while being continuously superfused (at 2 mL/min) with control solution containing (in mM): 152 NaCl, 5 KCl, 1 MgCl₂, 2 CaCl₂, 10 glucose, and 10 HEPES; pH was adjusted to 7.4 with NaOH and osmolarity was adjusted to 320 mOsM with glucose. Patch pipettes had a resistance of 3 to 4 MW when filled with intracellular solution containing (in mM): 130 CsCl, 0.5 CaCl₂, 5 MgCl₂, 5 K₂ATP, 0.5 NaGTP, 10 HEPES and 5 EGTA; pH was adjusted to 7.2 with CsOH. Responses to selective P2X3 receptor agonist α,β-methylene-ATP (α,β-meATP; resistant to ectoATPase hydrolysis, Sigma-Aldrich) were measured using an EPC-9 amplifier and HEKA Patch Master software (HEKA Electronik, Germany). Cells were voltage-clamped at −70 mV. In most cells, series resistance was compensated by 80%. To determine half effective concentration of agonist (EC₅₀), dose-response curves for α,β-meATP were constructed by applying different agonist doses to the same cells and fitting them with a logistic equation (Origin 8.0, Microcal, Northampton, Mass.).

### Drug Delivery

Agonists and antagonists were applied, usually for 2 sec, via a rapid superfusion system (Rapid Solution Changer RSC-200; BioLogic Science Instruments, Grenoble, France) placed 100 to 150 μm near the cell. The time for the solution exchange across the cell was approximately 30 ms, as judged with liquid junction potential measurements. All chemicals, including enzymes for cell culture, were from Sigma (St. Louis, Mo.). Culture media were obtained from Invitrogen (Milan, Italy).

### Data Analysis

The peak amplitudes of the responses were measured using HEKA Patch Master software. For each agonist, dose-response plots were constructed by normalizing data with respect to the maximum response. All data are presented as mean±S.E.M. (n=the number of cells) with statistical significance assessed by paired t-test (for parametric data) or Mann-Whitney rank sum test (for non-parametric data). Best fits of data with a sigmoid function were compared with respective control fits using Origin 8.0 software. A value of P<0.05 was accepted as indicative of statistically significant difference.

### Measurement of Nociceptive Behavior in Formalin Test

The formalin test experiments were performed on 21-day old male Wistar rats, weighing 40±5 g according to the method described previously (Simonetti et al, 2006; Yegutkin et al, 2008). Briefly, animal was acclimated to an acrylic observation chamber for at least 1 h before the injection of formalin (25 μL of 0.5% solution in 0.9% saline) into the dorsal surface of the right hindpaw. Age-matched control animals were injected with saline (100 μL). Immediately after the injection, each animal was returned to the observation chamber and its behavioural characteristics were recorded for 45 min. We noticed consistently observed type of response: spontaneous jerking of the injected paw. The number of jerks was measured for each 5-mM block, using self-developed computer software.

### Example 1

### Agonist Activity of AppNHppA Acting on Rat Homomeric P2X3 Receptors

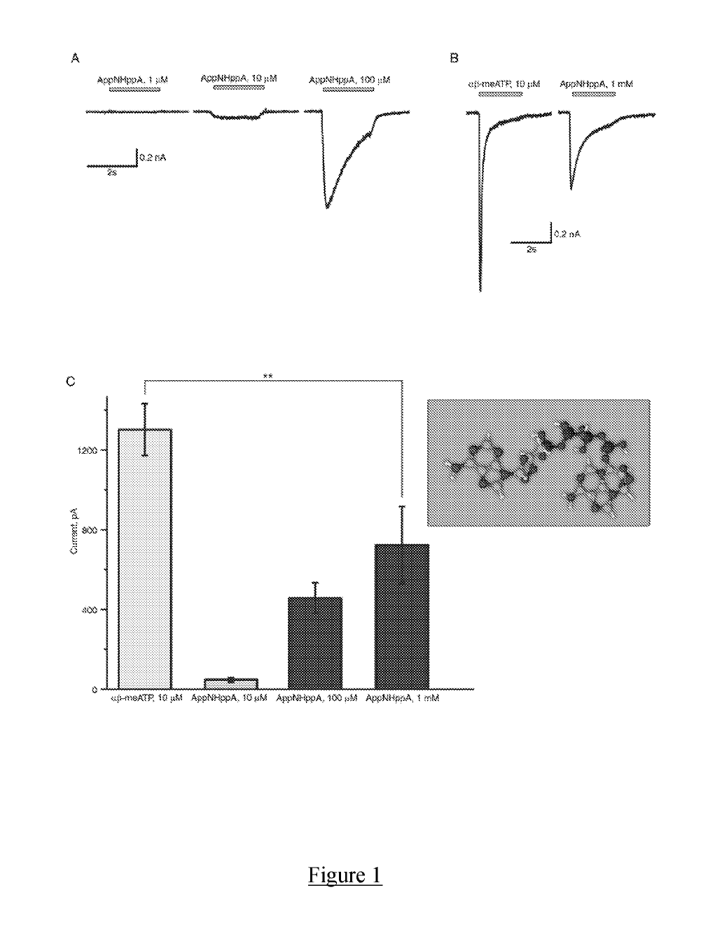

In experiments using HEK cells in vitro expressing rat homomeric P2X3 receptors, the analogue AppNHppA was found not to induce membrane currents at concentrations of 0.1 or 1 μM. However, small and slowly desensitizing currents were generated by 10 or 100 μM AppNHppA (FIG. 1A).

To test whether AppNHppA is a full or partial agonist of P2X3 receptors, the inventors compared induced responses with those from full P2X3 agonist α,β-meATP (FIG. 1B). In the same cell, they observed that the current response elicited by AppNHppA even at a saturating 1 mM concentration was 41.2±13% (n=3, P=0.009 by paired t-test) of the response induced by 10 μM of α,β-meATP (FIG. 1C). These results indicate that AppNHppA is only a partial agonist activity of homomeric P2X3 receptors at high concentrations.

### Example 2

### Inhibitory (Down-Regulatory) Effects of AppNHppA and AppCH₂ppA on P2X3 Receptor Activity

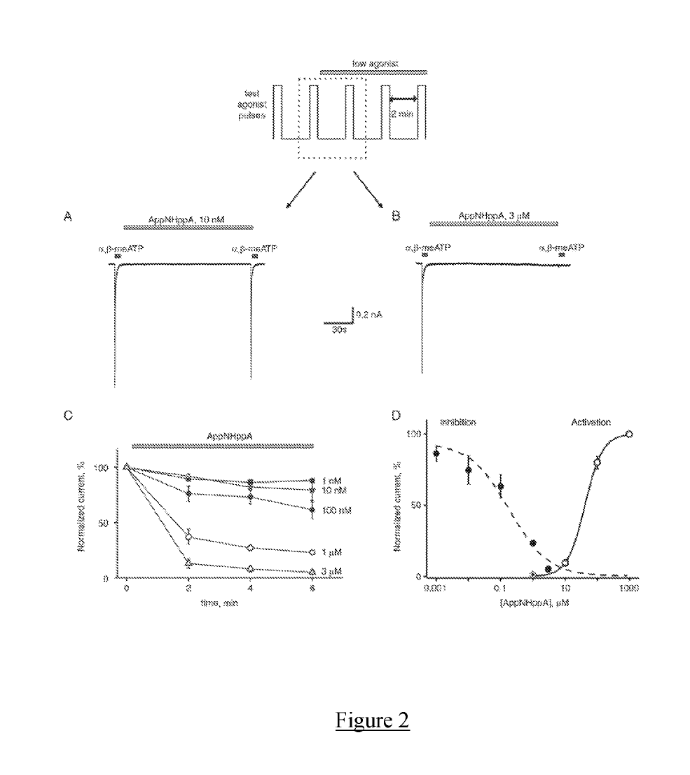

The inhibitory (suppressive) potencies of AppNHppA and AppCH₂ppA via high affinity desensitization (HAD) were evaluated. The inventors applied both synthetic analogues in different experiments during rhythmic 2 s long (2 mM interpulse interval) activations of P2X3 receptors using 10 μM α,β-meATP (for protocol see FIG. 2 top). When 10 nM AppNHppA was applied for 6 mM after the second test pulse of α,β-meATP, HAD did not appear to be induced since the amplitude of the following response was almost unchanged (FIG. 2A). However when 3 μM AppNHppA was administered, a strong (almost full) inhibition (suppression) was induced on test responses (FIG. 2B). Notably, when AppNHppA was administered at a concentration below the activation threshold (100 nM; see FIG. 2D), then a strong HAD response was induced with test responses reduced to 38.2±8.5% (n=7, P=0.002). The time course for these inhibitory effects of AppNHppA is also shown (FIG. 2C) and the concentration dependence measured at saturation (6 mM after drug application) demonstrates an IC₅₀ value of 0.20 μM (FIG. 2D). Importantly, the inhibitory curve for AppNHppA did not overlap with the activation curve, since the activation of P2X3 receptors mediated by AppNHppA appeared to require much higher concentrations of analogue (EC₅₀=41.6±1.3 μM; n_{H}=1.58±0.05; n=8; FIG. 2D).

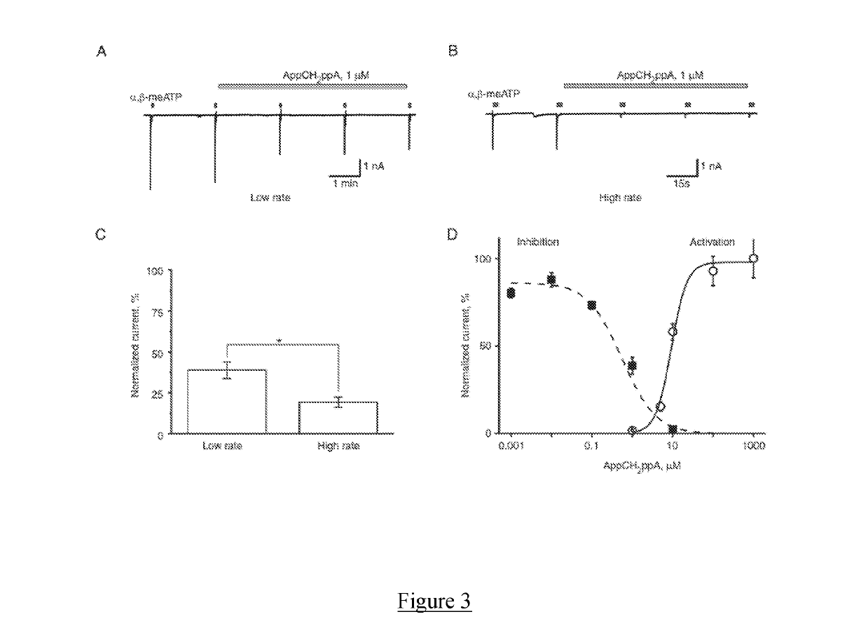

In parallel to these studies, the inhibitory (down-regulatory) effects of AppCH₂ppA were also investigated (IC₅₀=0.55 μM) with 2 min test applications of 10 μM α,β-meATP. The effects of administering AppCH₂ppA were similar to the effects of administering AppNHppA (FIG. 3), although the agonist effect was stronger (EC₅₀=9.30±1.7 μM; n_{H}=2.27±0.56; n=5; FIG. 3D). It should also be noted that with an increased frequency of agonist applications (30 s intervals; FIG. 3B,C) when the fraction of P2X3 receptor in a desensitised state was increased, then the inhibitory (down-regulatory) effect of AppCH₂ppA was also significantly increased (depression by 81±3% vs 61.3±5% at low rate, P=0.014, n=7) consistent with a use-dependent depressant effect on P2X3 receptors, operating via the HAD mechanism.

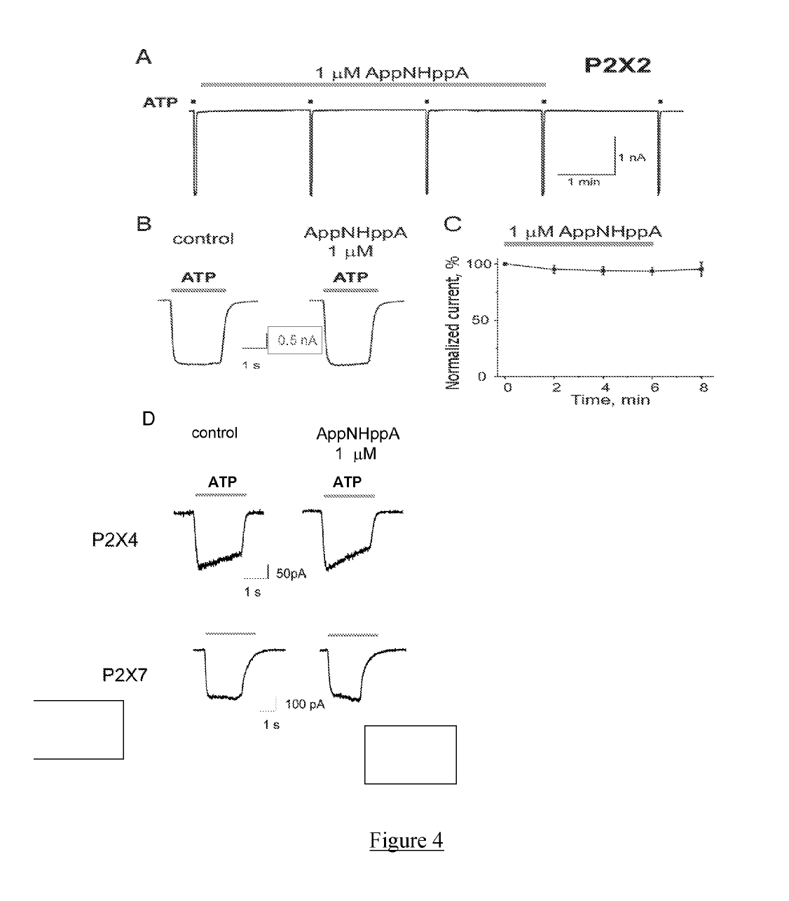

In a separate set of experiments with recombinant homomeric P2X2 receptors expressed in HEK 293 cells, the application of AppNHppA was not observed to produce any effects on P2X2 receptor-mediated currents (or on P2X4 or P2X7) hence demonstrating selectivity in our observed Ap₄A analogue effects (FIG. 4).

Altogether these data indicate that analogues of Ap₄A induce selective inhibition (down-regulation) of recombinant P2X3 receptors due to stabilization of the receptors in a desensitized state.

### Example 3

### Modulation of Native P2X3 Receptors in Rat Cultured Sensory Neurons

The action of AppNHppA was tested on α,β-meATP-induced currents separately in trigeminal ganglia (TG), dorsal root ganglia (DRG) and nodose ganglia (NG) neurons. The inhibitory effect of AppNHppA administered at 1 μM was evaluated using all three ganglia neurons.

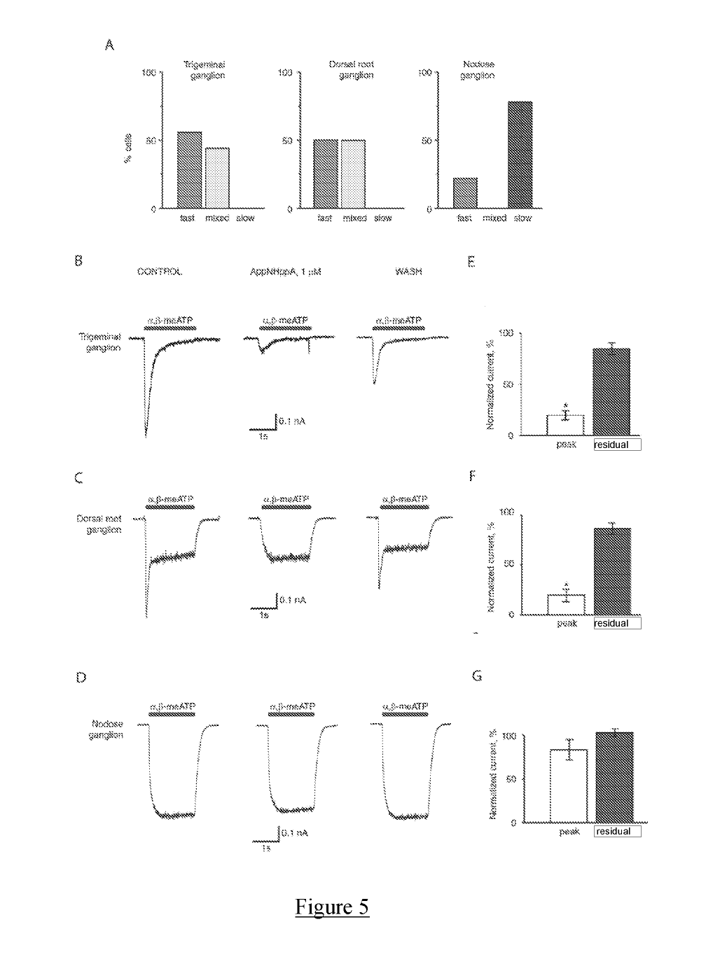

The time course of responses to ATP in situ strongly depends on differential contributions of P2X2 and P2X3 subunits. Fast desensitizing (fast) responses presumably reflect a P2X3 receptor contribution while sustained (slow) and composite (mixed) responses reflect a contribution from P2X2R and/or heteromeric P2X2P2X3 receptors. Upon application of α,β-meATP the proportion of cells displaying fast, mixed and slow responses differed depending on neuron cell populations. With TG neurons α,β-meATP induced mainly fast (56% of the cells) and mixed (44% of the cells) but no slow type responses (0% of the cells, n=18 cells). The proportion of cells with fast, mixed and slow responses was 50%, 50%, and 0% in DRG (n=8) and 22%, 0%, 78% (n=11) in NG neurons (FIG. 5A). Administration of AppNHppA (1 μM) selectively inhibited (suppressed) fast responses (FIG. 5B, C) with only a modest inhibitory effect on sustained responses (FIG. 5D) Inhibition (suppression) of the peak current was 80.3±4.4% (n=9, P=0.0004) in TG, 79.2±5.6% (n=6, P=0.02) in DRG and only 16.8±11.8% (n=9, P>0.05) in NG neurons (FIG. 5E-G). This is in line with the relatively larger proportion of cells with fast currents mediated by P2X3R subunits in populations of TG and DRG neurons. Consistent with this findings, sustained residual components were less inhibited (inhibition was 15±6%, 18±6% and 2±4% in TG, DRG and NG neurons, respectively; FIG. 5E-G). Hence, data on native neurons are consistent with the results obtained with recombinant homomeric P2X3 receptor.

### Example 4

### Antinociceptive Effects of Hydrolysis Resistant Ap₄a Analogues in Formalin Test

The effects of AppNHppA and AppCH₂ppA were examined on the behavioral reactions of rats in inflammatory pain models. Injection of diluted formalin into a rodent's hindpaw produces a biphasic nociceptive response consisting of immediate (acute phase) and tonic (inflammatory phase) components. The first phase of a formalin response (i.e. 0-5 min after injection) involves a direct effect of formalin on the pain receptors, while changes in animal behavior during the second tonic phase (i.e. 7-45 min after injection) are caused by hyperalgesia that develops due to the sensitization of nociceptive and spinal neurons via mechanisms that are triggered by repetitive stimulation during the first phase.

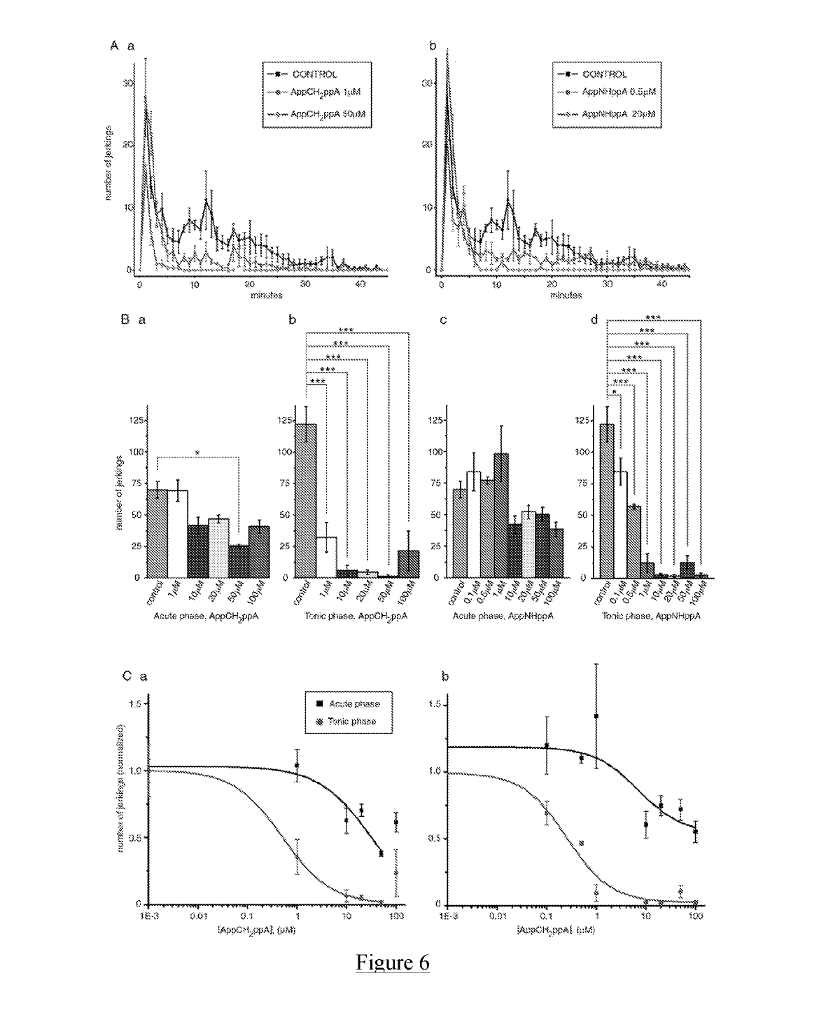

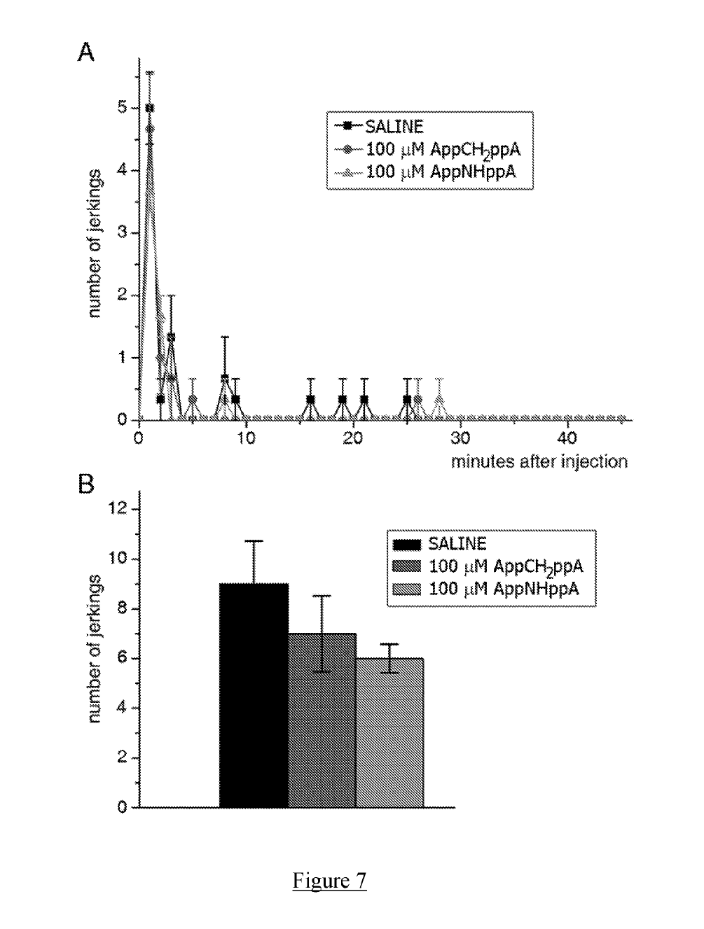

Co-injection of AppNHppA or AppCH₂ppA (100 μL) and formalin into the inflamed paws of rats potently reduced the number of nocifensive events triggered by the injection of formalin. The IC₅₀ value for AppNHppA in the second (tonic) phase was 0.25±0.06 μM, k_{H}=0.89±0.18, while the IC₅₀ value in the first (acute) phase was >100 μM; in comparison, the IC₅₀ value for AppCH₂ppA in the second phase was 0.5±0.06 μM, k_{H}=0.89±0.18, whilst the IC₅₀ value in the first phase was found to be 31.9±22 μM; k_{H}=0.82±0.09. Specifically post AppCH₂ppA administration, nocifensive behavior was reduced 60-times more effectively in the second (tonic) phase compared with the first (acute) phase of the formalin assay (FIG. 6). Under control conditions no signs of nociceptive behaviour were observed after injection of compounds themselves (FIG. 7).

In another related experiment, A_{diol}ppCH₂ppA_{diol} and AppCH₂ppG have been tested for their antinociceptive ability in a Formalin pain test.

Data are summarized in the Table 1 below, with IC₅₀ expressed as concentrations and as calculated nmolkg.

TABLE 1

| | First phase (acute pain) | | | | Second phase (chronic pain) | | | |
|---|---|---|---|---|---|---|---|---|
| A_{diol}ppCH₂ppA_{diol} | ~50 | μM | ~125 | nmol/kg | 0.084 ± 0.14 | μM | 0.21 ± 0.35 | nmol/kg |
| AppCH₂ppG | >100 | μM | >250 | nmol/kg | 0.18 ± 0.12 | μM | 0.45 ± 0.30 | nmol/kg |
| AppNHppA | >100 | μM | >250 | nmol/kg | 0.25 ± 0.09 | μM | 0.63 ± 0.23 | nmol/kg |
| AppCH₂ppA | 32 ± 2.2 | μM | 80 ± 5.5 | nmol/kg | 0.51 ± 0.06 | μM | 1.28 + 0.06 | nmol/kg |

Indeed, recalculation of the effective concentrations of AppCH₂ppA and AppNHppA assuming 40 g rat body weight, respectively 1.28±0.06 nmolkg and 0.63±0.23 nmolkg, clearly demonstrate that the compounds of the present invention are about 5,000 times more potent than other P2X3 antagonists such as A-317491 (Jarvis et al., 2002).

As a calculation from a rat's weight, being about 40 g, to average human's weight, being about 70 kg, the approximate indicative dose of compound to be administered to a human being in need of treatment could be estimated as shown in Table 2.

TABLE 2

| | First phase (acute pain) | | | Second phase (chronic pain) | | |
|---|---|---|---|---|---|---|
| | nmol/kg | Rat (40 g) | Human (70 kg) | nmol/kg | Rat (40 g) | Human (70 kg) |
| | (μg/kg) | nmol | nmol | (μg/kg) | nmol | nmol |
| A_{diol}ppCH₂ppA_{diol} | ~125 | 5 | 8750 | 0.21 ± 0.35 | 0.0084 | 14.7 |
| | (104) | | | (0.175) | | |
| AppCH₂ppG | >250 | 10 | 17500 | 0.45 ± 0.30 | 0.018 | 31.5 |
| | (209) | | | (0.376) | | |
| AppNHppA | >250 | 10 | 17500 | 0.63 ± 0.23 | 0.0252 | 44.1 |
| | (209) | | | (0.526) | | |
| AppCH₂ppA | 80 ± 5.5 | 3.2 | 5600 | 1.28 ± 0.06 | 0.0512 | 89.6 |
| | (67) | | | (1.067) | | |

### Example 5

### Antinociceptive Effects of Hydrolysis Resistant Ap₄a Analogues in Hargreaves Test

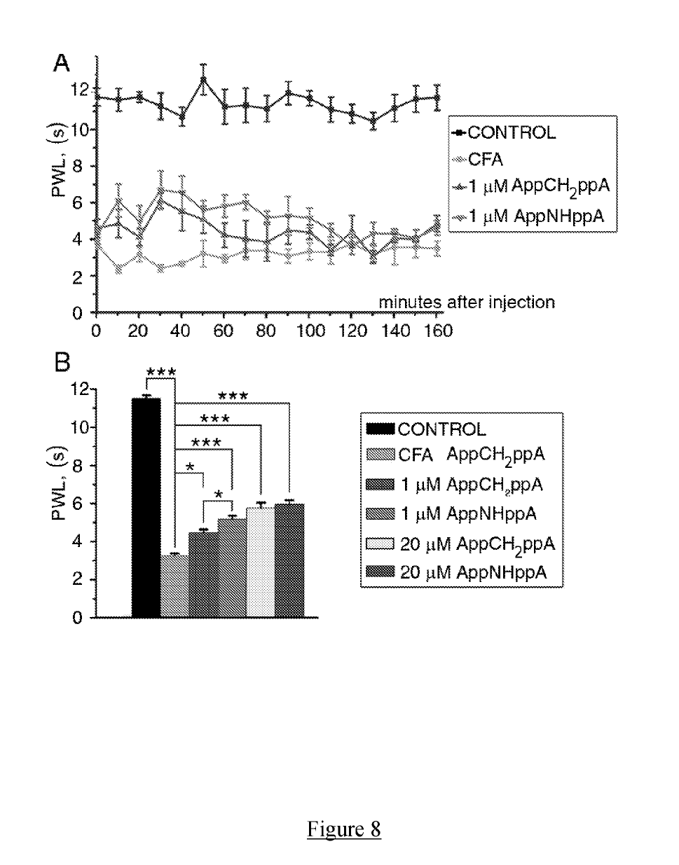

In the Hargreaves plantar test, the antinociceptive effect was observed following intraplantar injection of AppNHppA and AppCH₂ppA (100 μL) into the inflamed hind paw of rats under complete Freund adjuvant-induced thermal hyperalgesia (FIG. 8).

### Example 6

### Antinociceptive Effect of AppCH₂ppA in a Partial Sciatic Nerve Ligation (PSNL) Test

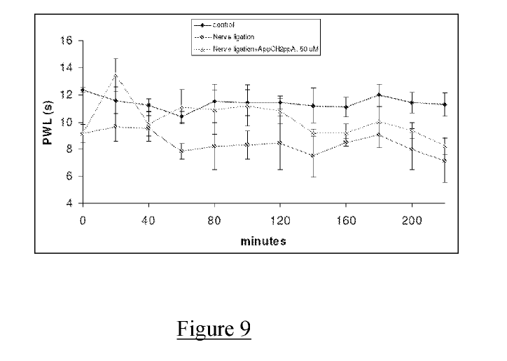

PSNL, with tight ligation of 33-50% of the sciatic nerve trunk, is a neuropathic pain model. We sub-cutaneously injected 50 μM of AppCH₂ppA (100 pt) on thermal hyperalgesia in rats with PSNL. FIG. 9 shows the time-course measurements of paw withdrawal latency (PWL), and demonstrates the antinociceptive and antineuropathic effect of the compound. Results are expressed as mean+/−S.E.M. In this model, the antinociceptive effect is 62%.

### Example 7

### Compared Antinociceptive Effect of Intrathecal and Intraplantar Administration of AppCH₂ppA

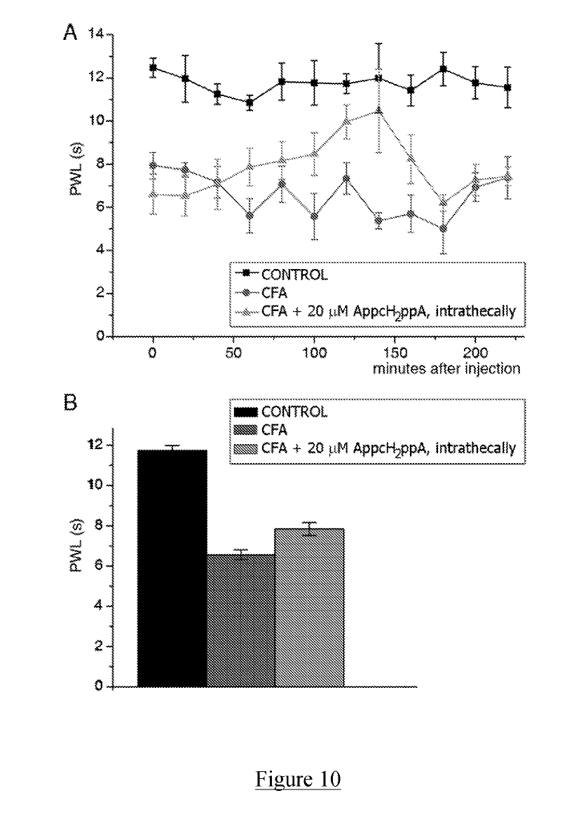

In a Hargreaves plantar test under complete Freund adjuvant (CFA), the intrathecal injection of 20 μM of AppCH₂ppA demonstrated a weaker delayed antinociceptive effect than observed after intraplantar option. FIG. 10A shows the time course of the effects of intra-thecal injection of AppCH₂ppA on CFA-induced thermal hyperalgesia. FIG. 10B shows a bar-graph representation of the effects of intra-thecal administration of AppCH₂ppA on rat PWL.

Respectively 27% and 53% of antinociceptive effect were measured after intra-thecal and intraplantar administration. These data are consistent with the local peripheral action of the compounds and with the fact that the effects are not mediated by P2X3 receptors expressed in CNS.

A key advantage of targeting P2X3 receptors is that they have limited distribution beyond sensory nerves and no significant expression in the higher centres of the brain. Based on this, it would be expected that the compound would not elicit bothersome or serious CNS side effects that otherwise limit substantially the utility of many pain treatments. It seems very important that Ap₄A analogues prevent activation of nociceptive pathways peripherally, thus preventing transmission of pain signals to downstream synapses leading to the development of pathologic plasticity and sensitization in higher structures that could contribute to the amplification of pain signals in chronic pain.

In the present experiments, local peripheral intraplantar injection induced two-fold higher effect than observed following intrathecal injection. These data are consistent with the involvement of a peripheral P2X3 mediated mechanism.

All publications mentioned in the above specification are herein incorporated by reference. Various modifications and variations of the described methods and systems of the invention will be apparent to those skilled in the art without departing from the scope and the spirit of the invention. Although the invention has been described in connection with specific preferred embodiments, it should not be understood that the invention as claimed should not be unduly limited to such specific embodiments. Indeed, various modifications of the described modes for carrying out the invention which are obvious to those skilled in chemistry, biology or related fields are intended to be within the scope of the following claims.

### REFERENCES

Fabbretti E, Sokolova E, Masten L, D'Arco M, Fabbro A, Nistri A, Giniatullin R: Identification of negative residues in the P2X3 ATP receptor ectodomain as structural determinants for desensitization and the Ca2+-sensing modulatory sites. J Biol Chem 2004, 279:53109-53115.
Gever J R, Cockayne D A, Dillon M P, Burnstock G, Ford A P: Pharmacology of P2X channels. Pflugers Arch 2006, 452:513-537.
Gever J R, Soto R, Henningsen R A, Martin R S, Hackos D H, Panicker S, Rubas W, Oglesby I B, Dillon M P, Milla M E, Burnstock G, Ford A P: AF-353, a novel, potent and orally bioavailable P2X3P2X23 receptor antagonist. British journal of pharmacology 2010, 160: 1387-1398.
Grishin E V, Savchenko G A, Vassilevski A A, Korolkova Y V, Boychuk Y A, Viatchenko-Karpinski V Y, Nadezhdin K D, Arseniev A S, Pluzhnikov K A, Kulyk V B, et al: Novel peptide from spider venom inhibits P2X3 receptors and inflammatory pain. Ann Neurol, 67:680-683.
Jarvis M F, Burgard E C, McGaraughty S, Honore P, Lynch K, Brennan T J, Subieta A, Van Biesen T, Cartmell J, Bianchi B, et al: A-317491, a novel potent and selective non-nucleotide antagonist of P2X3 and P2X23 receptors, reduces chronic inflammatory and neuropathic pain in the rat. Proc Natl Acad Sci USA 2002, 99:17179-17184.
McDonald H A, Chu K L, Bianchi B R, McKenna D G, Briggs C A, Burgard E C, Lynch K J, Faltynek C, Cartmell J, Jarvis M F: Potent desensitization of human P2X3 receptors by diadenosine polyphosphates. Eur J Pharmacol 2002, 435:135-142.
McGaraughty S, Wismer C T, Zhu C Z, Mikusa J, Honore P, Chu K L, Lee C H, Faltynek C R, Jarvis M F: Effects of A-317491, a novel and selective P2X3P2X23 receptor antagonist, on neuropathic, inflammatory and chemogenic nociception following intrathecal and intraplantar administration. British journal of pharmacology 2003, 140: 1381-1388.
Melnik S, Wright M, Tanner J A, Tsintsadze T, Tsintsadze V, Miller A D, Lozovaya N: Diadenosine polyphosphate analog controls postsynaptic excitation in CA3-CA1 synapses via a nitric oxide-dependent mechanism. J Pharmacol Exp Ther 2006, 318:579-588.
Simonetti M, Fabbro A, D'Arco M, Zweyer M, Nistri A, Giniatullin R, Fabbretti E: Comparison of P2X and TRPV1 receptors in ganglia or primary culture of trigeminal neurons and their modulation by NGF or serotonin. Mol Pain 2006, 2:11.
Sokolova E, Nistri A, Giniatullin R: Negative cross talk between anionic GABAA and cationic P2X ionotropic receptors of rat dorsal root ganglion neurons. J Neurosci 2001, 21:4958-4968.
Sokolova E, Skorinkin A, Fabbretti E, Masten L, Nistri A, Giniatullin R: Agonist-dependence of recovery from desensitization of P2X(3) receptors provides a novel and sensitive approach for their rapid up or downregulation. Br J Pharmacol 2004, 141:1048-1058.
Wildman S S, Brown S G, King B F, Burnstock G: Selectivity of diadenosine polyphosphates for rat P2X receptor subunits. Eur J Pharmacol 1999, 367:119-123.
Wright M, Tanner J A, Miller A D: Quantitative single-step purification of dinucleoside polyphosphates. Anal Biochem 2003, 316:135-138.
Wright M, Miller A D: Synthesis of novel fluorescent-labelled dinucleoside polyphosphates. Bioorg Med Chem Lett 2004, 14:2813-2816.
Wright M, Miller A D: Novel fluorescent labelled affinity probes for diadenosine-5′,5′″-P1,P4-tetraphosphate (Ap4A)-binding studies. Bioorg Med Chem Lett 2006, 16:943-948.
Yegutkin G G: Nucleotide- and nucleoside-converting ectoenzymes: Important modulators of purinergic signalling cascade. Biochim Biophys Acta 2008, 1783:673-694.

1. A method of inhibition or down-regulation of a pain transducing ATP-gated P2X3 receptor, the method comprising administering an effective amount of a dinucleoside polyphosphate analogue, or a pharmaceutically acceptable salt thereof.

2. The method according to claim 1 wherein said dinucleotide polyphosphate analogue:
a) acts only on, or is selective for only, the P2X3 receptor (out of the P2X family);
b) does not act on either the P2X4, P2X7 and/or P2X2 receptor(s);
c) acts via a high affinity desensitisation (HAD) inhibition mechanism; or
(d) is a partial agonist or super-agonist of the P2X3 receptor.

3. The method according to claim 1 wherein said dinucleotide polyphosphate analogue is a compound of formula (I): 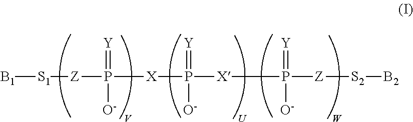
or a pharmaceutically acceptable salt thereof,
wherein X, X′ and Z are independently selected from 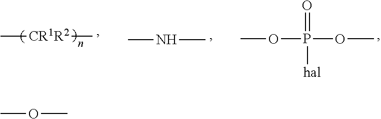
wherein R¹ and R² are independently selected from hydrogen, halogen, hydroxyl, cyano or an unsubstituted group selected from C₁₋₃ haloalkyl, C₁₋₃ alkyl, C₁₋₄ aminoalkyl and C₁₋₄ hydroxyalkyl, and n is selected from 1, 2, 3, 4, 5 and 6;
each Y is independently selected from ═S and ═O;
B₁ and B₂ are independently selected from a 5- to 7-membered carbon-nitrogen heteroaryl group which may be unfused or fused to a further 5- to 7-membered carbon-nitrogen heteroaryl group
S₁ and S₂ are independently selected from a bond, C₁₋₆ alkylene, C₂₋₆ alkenylene, C₂₋₆ alkynylene and a moiety of formula (II): 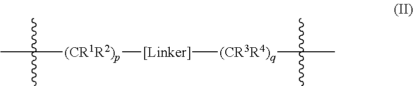
wherein
R¹, R², R³ and R⁴ independently represent hydrogen, halogen, hydroxyl, cyano or an unsubstituted group selected from C₁₋₃ haloalkyl, C₁₋₃ alkyl, C₁₋₄ aminoalkyl and C₁₋₄ hydroxyalkyl;
p and q independently represent 0, 1, 2 or 3, preferably 0, 1 or 2; and
[Linker] represents:
(i) —O—, —S—, —C═O— or NH—;
(ii) C₁₋₄ alkylene, C₂₋₄ alkenylene or C₂₋₄ alkynylene, which may optionally contain or terminate in an ether (—O—), thioether (—S—), carbonyl (—C═O—) or amino (NH—) link, and which are optionally substituted with one or more groups selected from hydrogen, hydroxyl, halogen, cyano, NR⁵R⁶ or an unsubstituted group selected from C₁₋₄ alkyl, C₂₋₄ alkenyl, C₁₋₄ alkoxy, C₂₋₄ alkenyloxy, C₁₋₄ haloalkyl, C₂₋₄ haloalkenyl, C₁₋₄ aminoalkyl, C₁₋₄ hydroxyalkyl, C₁₋₄ acyl and C₁₋₄ alkyl-NR⁵R⁶ groups, wherein R⁵ and R⁶ are the same or different and represent hydrogen or unsubstituted C₁₋₂ alkyl; or
(iii) a 5 to 7 membered heterocyclyl, carbocyclyl or aryl group, which may be optionally substituted with one or more groups selected from hydrogen, hydroxyl, halogen, cyano, NR⁵R⁶ or an unsubstituted group selected from C₁₋₄ alkyl, C₂₋₄ alkenyl, C₁₋₄ alkoxy, C₂₋₄ alkenyloxy, C₁₋₄ haloalkyl, C₂₋₄ haloalkenyl, C₁₋₄ aminoalkyl, C₁₋₄ hydroxyalkyl, C₁₋₄ acyl and C₁₋₄ alkyl-NR⁵R⁶ groups, wherein R⁵ and R⁶ are the same or different and represent hydrogen or unsubstituted C₁₋₂ alkyl;
V is selected from 0, 1, 2, 3, 4 and 5;
U is selected from 0, 1, 2, 3, 4 and 5;
W is selected from 0, 1, 2, 3, 4 and 5; and
V plus U plus W is an integer from 2 to 7.

4. The method according to claim 3, wherein in formula (I) B₁ and B₂ are independently selected from purine and pyrimidine nucleic acid bases.

5. The method according to claim 4, wherein in formula (I) B₁ and B₂ are independently selected from adenine, guanine, thymine, cytosine, uracil, hypoxanthine, xanthine, 1-methyladenine, 7-methylguanine, 2-N,N-dimethylguanine, 5-methylcytosine and 5,6-dihydrouracil.

6. (canceled)

7. (canceled)

8. The method according to claim 3 wherein S₁ and S₂ are independently selected from a bond, C₁₋₆ alkylene, C₂₋₆ alkenylene, C₂₋₆ alkynylene and a moiety of formula (III) or (IV): 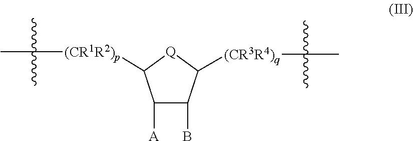
wherein
R¹, R², R³ and R⁴ independently represent hydrogen, halogen, hydroxyl, cyano or an unsubstituted group selected from C₁₋₃ haloalkyl, C₁₋₃ alkyl, C₁₋₄ aminoalkyl and C₁₋₄ hydroxyalkyl;
p and q independently represent 0 or 1;
Q represents —O—, —S—, —C═O—, —NH— or CH₂; and
A and B independently represent hydrogen, hydroxyl, halogen, or an unsubstituted group selected from C₁₋₄ alkoxy, C₁₋₄ aminoalkyl, C₁₋₄ hydroxyalkyl, C₁₋₄ acyl and —NR⁵R⁶ groups, wherein R⁵ and R⁶ are the same or different and represent hydrogen or unsubstituted C₁₋₂ alkyl; 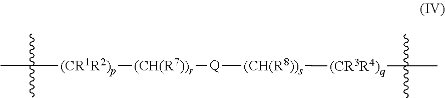
wherein
R¹, R², R³ and R⁴ independently represent hydrogen, halogen, cyano or an unsubstituted group selected from C₁₋₃ haloalkyl, C₁₋₃ alkyl, C₁₋₄ aminoalkyl and C₁₋₄ hydroxyalkyl;
Q represents —O—, —S—, —C═O—, NH— or CH₂; and
R⁷ and R⁸ independently represent hydrogen, hydroxyl, halogen, cyano, —NR⁵R⁶ or an unsubstituted group selected from C₁₋₄ alkyl, C₂₋₄ alkenyl, C₁₋₄ alkoxy, C₂₋₄ alkenyloxy, C₁₋₄ haloalkyl, C₂₋₄ haloalkenyl, C₁₋₄ aminoalkyl, C₁₋₄ hydroxyalkyl, C₁₋₄ acyl and C₁₋₄ alkyl-NR⁵R⁶ groups, wherein R⁵ and R⁶ are the same or different and represent hydrogen or unsubstituted C₁₋₂ alkyl; and
p, q, r and s independently represent 0 or 1.

9. (canceled)

10. (canceled)

11. The method according to claim 10 wherein in formula (I) S₁ and S₂ are D-ribofuranose or ring opened D-ribofuranose.

12. (canceled)

13. The method according to claim 3 wherein X and X′ are independently selected from NH and
—(CR¹R²)ₙ—,
preferably wherein R¹ and R² are both H and n is 1 or 2.

14. The method according to claim 3 wherein in formula (I) each Y is ═O and each Z is —O—.

15. The method according to claim 3 wherein said dinucleotide polyphosphate analogue is a compound of formula (I′): 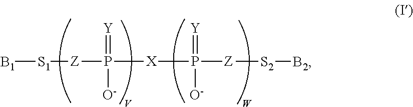
wherein X is not —O— and V plus W is an integer from 2 to 7.

16. The method according to claim 15 wherein in formula (I′) V plus W is 4 or 5.

17. (canceled)

18. (canceled)

19. The method according to claim 1 wherein said dinucleoside analogue is an Ap₄A or Ap₄G analogue chosen among the group consisting of: AppCH₂ppA, AppNHppA, A_{diol}ppCH₂ppA_{diol}, A_{diol}ppNHppA_{diol}, A_{diol}ppNHppA_{diol}, AppCH₂ppG, AppNHppG, A_{diol}ppCH₂ppG_{diol} and A_{diol}ppNHppG_{diol}:

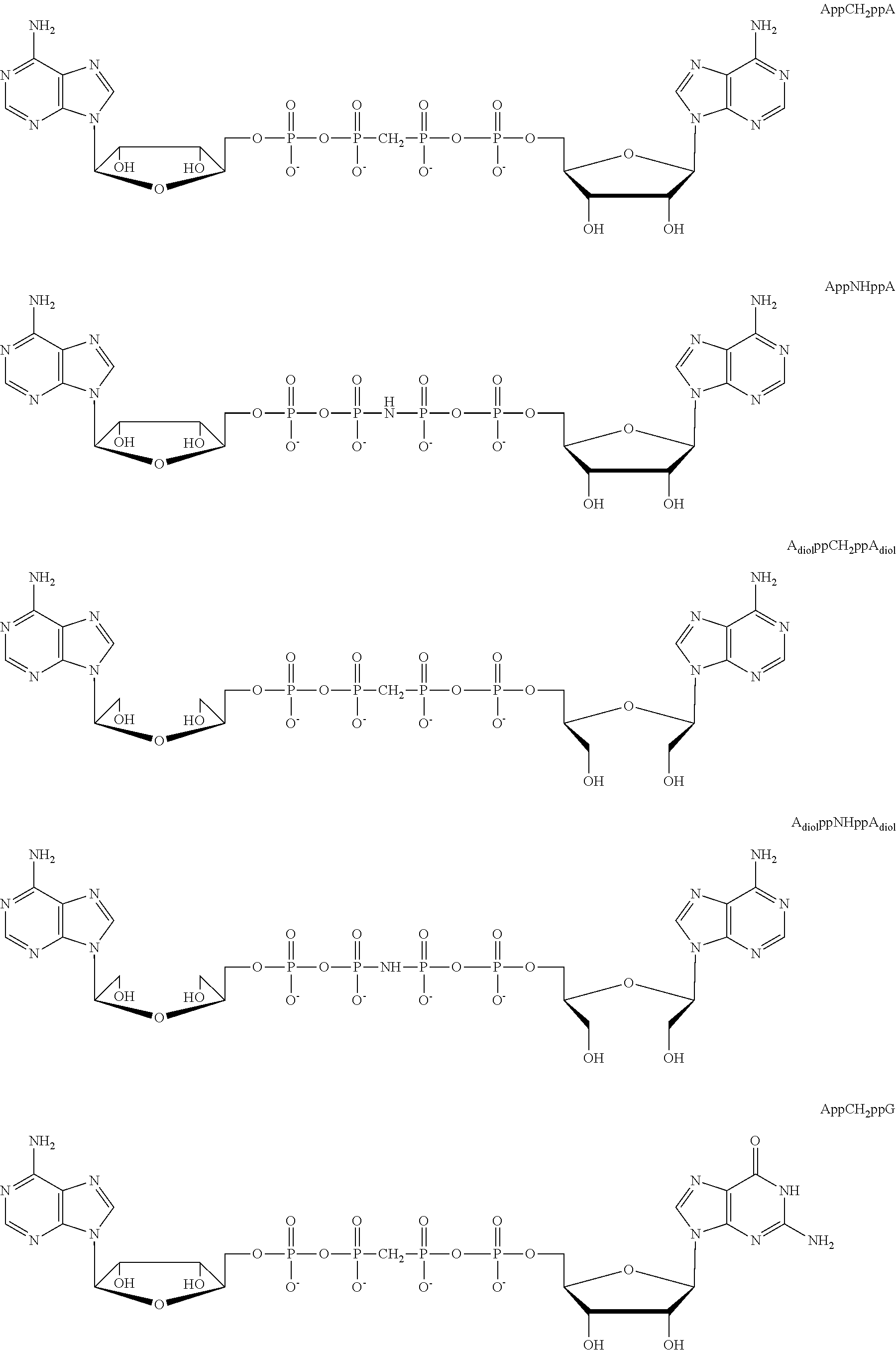

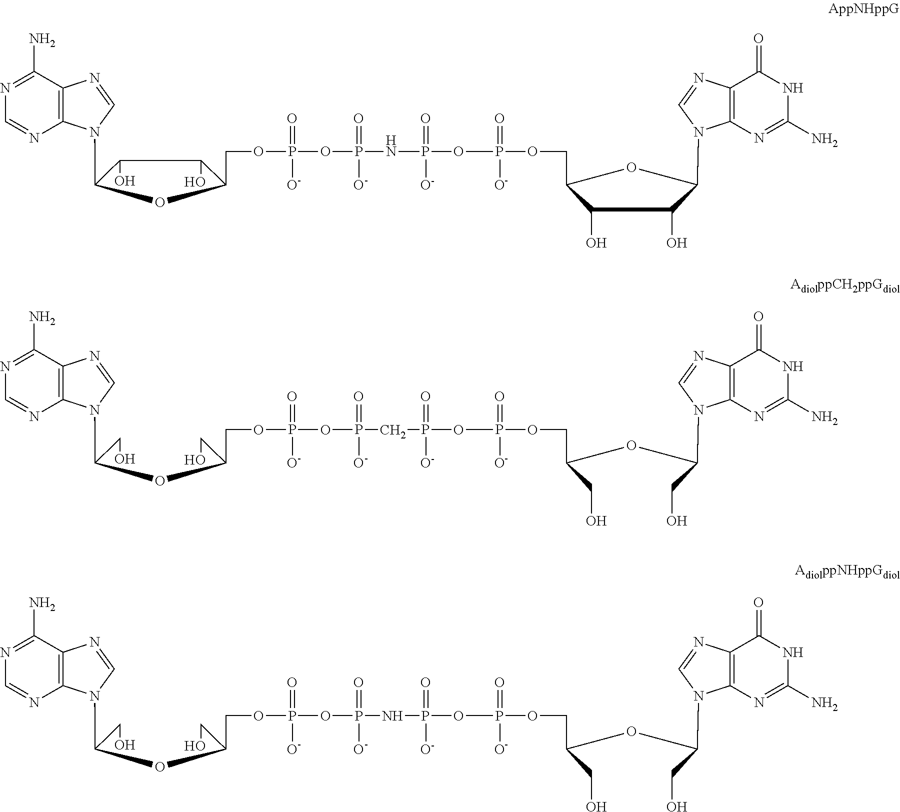

20. The method according to claim 1 for use in the treatment of pain.

21. (canceled)

22. The method according to claim 20, for use in the treatment of pain associated with one or more of inflammation, back pain, trapped nerve, arthritic pain, cancer-related pain, dental pain, endometriosis, birthing-related pain, post-surgical pain or trauma.

23. The method according to claim 20, wherein the pain is moderate to chronic pain.

24. (canceled)

25. The method according to claim 20, wherein the dinucleoside polyphosphate analogue is administered in an amount of 0.01 to 10 μg/kg.

26. The method according to claim 20, wherein the pain is acute pain or subacute pain.

27. The method according to claim 26, wherein the dinucleoside polyphosphate analogue is administered in an amount of 10 to 500 μg/kg.

28. The method according to claim 1, wherein the dinucleoside polyphosphate analogue is administered in combination with another pharmaceutically active agent.

29. (canceled)

30. (canceled)

31. (canceled)

32. A method of treating moderate to chronic pain or back pain, comprising administering an effective amount of a dinucleoside polyphosphate analogue of formula (I) as defined in claim 3, or a pharmaceutically acceptable salt thereof.

33. (canceled)

34. (canceled)

35. A compound, such as a dinucleoside phosphase analogue, which:
a) acts only on, or is selective for only, the P2X3 receptor (out of the P2X family);
b) does not act on either the P2X4, P2X2 and/or P2X7 receptor(s);
c) acts via a high affinity desensitisation (HAD) inhibition mechanism; or
d) is a partial agonist or super-agonist of the P2X3 receptor.

36. A compound according to claim 35 which has the formula NPₙN (where N represents a nucleoside moiety, P represents a phosphate group and n is from 2 to 7).

37. A composition comprising a dinucleoside polyphosphate analogue, or a
pharmaceutically acceptable salt thereof, in an amount of from 0.01 to 3500 μg and a
pharmaceutically acceptable excipient.

38. (canceled)

39. A composition according to claim 37, wherein the dinucleoside polyphosphate analogue is as defined in claim 3.

40. (canceled)

41. (canceled)
